(19) United States
(12) Patent Application Publication
(10) Pub. No.: US 2019/0285635 A1
(43) Pub. Date: Sep. 19, 2019

(54) CNPY2 ISOFORM 2 AND THE USE THEREOF AS MOLECULAR MARKER FOR COLORECTAL CANCER

(71) Applicant: Shenzhen Senboll Biotechnology Co., LTD., Shenzhen (CN)

(72) Inventors: Jian GUO, Toronto (CA); Desen WAN, Guangzhou (CN); Yujing FANG, Guangzhou (CN); Qingian OU, Guangzhou (CN)

(21) Appl. No.: 16/464,261

(22) PCT Filed: Dec. 9, 2016
(86) PCT No.: PCT/CN2016/109159

§ 371 Date: May 24, 2019

(51) Int. Cl. G01N 33/574; C07K 16/18; C12Q 1/6886

(52) U.S. Cl. CPC G01N 33/57419; C07K 16/18; C12Q 1/6886; C12Q 2600/118; C07K 2317/33; C12Q 2600/158; G01N 2333/4703

(57) ABSTRACT

The present disclosure provides a molecular marker CNPY2 isoform 2 for colorectal cancer, which has the sequence identified herein as SEQ ID NO:1. The present disclosure also provides a specific antibody of CNPY2 isoform 2, a kit comprising said specific antibody and a medication for the treatment of colorectal cancer. Further, the present disclosure also provides a method for detection, diagnosis and prognosis of colorectal cancer and a method for prevention, alleviation and treatment of this cancer.

### FIELD OF THE INVENTION

This invention relates to the molecular biological detection, and specifically to CNPY2 isoform 2, a molecular marker of intestinal cancer. Further, the present invention relates to a specific antibody of CNPY2 isoform 2, a drug for treating intestinal cancer and a kit for diagnosing intestinal cancer, and methods for diagnosis and treatment of this cancer.

### BACKGROUND

With the improvement of living standard and change in diet, the incident of intestinal cancer in urban areas showed an upward trend year by year. Currently, colorectal cancer (CRC) has become the second leading cause of death among diseases in China. Comparing with many other cancers with high mortality, the cure rate of intestinal cancer is up to 90% if it is found and treated early, while only 10% in advanced patients. Intestinal cancer discovered and diagnosed at early stages can be cured by surgery, radiotherapy or chemotherapy.

At present, enteroscope is the only reliable diagnostic method for intestinal cancer, which however, requires cleansing bowel three days in advance and general anesthesia on the day of enteroscope, bringing a lot of pain and discomfort to the patients. Before an obvious clinical symptom such as hematochezia, there are very few people that ask for enteroscope, while when the hematochezia appears, the ideal time for treatment has passed.

Thus, if a diagnostic molecular marker, such as a secretory protein of one or several genes, can be developed, the risk of intestinal cancer will be able to be predicted with simple detection of 1 mL serum before painful and uncomfortable enteroscope according to the differential expression of the protein in the blood of normal humans and patients with intestinal cancer. Afterwards, further diagnosis by enteroscope is followed, with surgery, radiotherapy or chemotherapy at early stages. This kind of molecular marker is an important part of the Precision Medicine, which has immeasurably broad market prospects and will makes contribution to human health.

Doctor Guojian, one of the inventors of the present application, discovered a new gene named CNPY2 (canopy FGF signaling regulator 2) during his over six years as a senior researcher in the University of Toronto. The gene has two isoforms, isoform 1 and isoform 2, which are capable of being translated into two proteins with different length and sequence. The Genbank accession number of isoform 1 is NM_014255, which has 182 amino acids; and that of isoform 2 is NM 001190991, with only 84 amino acids. Isoform 2 is very short, which is predicted to be about 9 KDa theoretically.

In the preliminary research in University of Toronto, Dr. Guo found that isoform 1, the secretory protein product of the new gene, is a growth factor which promotes angiogenesis. Four high-level papers have been published according to this finding, and other three are under review. As compared to normal mice, mice over-expressing this gene are strong, while gene-knockout ones have small body size. Almost 20% mice die early during embryogenesis, and survived ones only have about half of the weight of normal mice (data unpublished) and suffer premature aging. This new gene has significantly increased expression under hypoxia, and published data have proved that the promoter of the gene is regulated by hypoxia inducible factor HIF-1α, which is consistent with some known growth factors.

Interestingly, Dr. Guo and one of his students in the laboratory of U of T occasionally found that, with more than twenty samples, isoform 1 of this new gene has much higher expression in the blood of colorectal cancer patients than that of normal humans. Through immunohistochemistry using self-made polyclonal antibody, the tissues of intestinal cancer are stained much more deeply than adjacent normal tissues, and decreasing expression of the new gene in cell level results in growth block and apoptosis of the intestinal cancer cells. The results have been published in American Journal of Pathology at the beginning of 2016 (Decreasing CNPY2 Expression Diminishes Colorectal Tumor Growth and Development through Activation of p53 Pathway. Yan P, Gong H, Zhai X, Feng Y, Wu J, He S, Guo J, Wang X, Guo R, Xie J, Li R K. Am J Pathol. 2016 Feb. 3. pii: S0002-9440(16)00011-0. doi: 10.1016/j.ajpath.2015.11.012. [Epub ahead of print]PMID: 26835537 (Impact Factor 5). Website: http://www.ncbi.nlm.nih.gov/pubmed/26835537).

The other related references in the cardiovascular field about this new gene CNPY2 isoform 1 are as follows:
Guo J, Mihic A, Wu J, Zhang Y, Singh K, Dhingra S, Weisel R D, Li R K. Canopy 2 attenuates the transition from compensatory hypertrophy to dilated heart failure in hypertrophic cardiomyopathy. Eur Heart J. 2015 Jul. 9. pii: ehv294. [Epub ahead of print] PMID: 26160001 (Impact Factor 17). Website: http://www.ncbi.nlm.nih.gov/pubmed/?term=26160001
(2) Guo J, Zhang Y, Mihic A, Li S H, Sun Z, Shao Z, Wu J, Weisel R D, Li R K. A Secreted Protein (Canopy 2, CNPY2) Enhances Angiogenesis and Promotes Smooth Muscle Cell Migration and Proliferation. Cardiovasc Res. 2015 Jan. 14. pii: cvv010. [Epub ahead of print] PMID: 25589425 (Impact Factor 7). Website: http://www.ncbi.nlm.nih.gov/pubmed/?term=25589425
(3) Hatta K, Guo J, Ludke A, Dhingra S, Singh K, Huang M L, Weisel R D, Li R K. Expression of CNPY2 in mouse tissues: quantification and localization. PLoS One. 2014 Nov. 13; 9(11):e111370. doi: 10.1371/journal.pone.0111370. eCollection 2014. PMID: 25393402 (Impact Factor 3.7). Website: http://www.ncbi.nlm.nih.gov/pubmed/?term=25393402

Some currently known growth factors, such as vascular endothelial growth factor VEGF, are expressed more highly in cancer cell tissues than in normal humans, and many of them are used by large biopharmaceutical companies abroad as target genes for treating cancers. However, the relationship between CNPY2 and tumors is still a blank, and there is no scientific reference reporting the relationship of CNPY2 with intestinal cancer or other tumors.

An ideal intestinal cancer gene molecular marker is supposed to be a serum protein product easy to operate, being cheap and bringing no pain or discomfort to patients, which meanwhile can be used to predict intestinal cancer lesion at the early stage of the cancer. Thus, the protein of this gene has to be expressed highly in intestinal cancer tissues and also must be secretory so as to be secreted into blood, and by simply drawing 1 mL blood, the protein can be detected by an antibody. Enzyme-Linked ImmunoSorbent Assay, ELISA, is an ideal detective method easy to operate and low in cost. As compared to other gene molecular detection methods, such as PCR or sequencing at DNA or RNA level, it has higher stability and repeatability and also lower cost.

Dr. Guo has kept researching the new CNPY2 in U of T for more than 6 years, and related papers published are all about CNPY2 isoform 1 (NM_014255). There is no reference or patent involved in CNPY2 isoform 2 (NM_001190991) that has been published or applied by scientific institutions or biotechnology companies worldwide.

That is, no biotechnology company or scientific institution so far is researching isoform 2 (NM_001190991) of this new CNPY2. All of the followings on CNPY2 isoform 2 remain unknown: its molecular biological function, and monoclonal and polyclonal antibodies thereagainst; whether it is a secretory protein and can detected by antibodies in the human serum; as a secretory protein ligand, it is supposed to combine with an unknown receptor on the cell surface, so as to deliver cell pathway signals, activate kinases downstream, and accelerate division and growth of (intestinal) cancer cells; whether a blocking monoclonal antibody inhibiting the binding of the ligand to receptor can be used clinically to treat intestinal cancer or other malignant tumors; and so on.

According to this, the inventor Dr. Guo concentrated on studying protein isoform 2 (NM_001190991) of this new gene CNPY2 in order to answer the above questions, develop its potential value, make it a new and accurate molecular marker for early diagnosis of intestinal cancer and explore related uses.

### SUMMARY OF THE INVENTION

To overcome the difficulty in diagnosis (particularly early diagnosis) of intestinal and related cancers, the present application aims to provide an ideal intestinal cancer gene molecular marker, a serum protein product easy to operate, low in cost, and bringing no pain or discomfort to patients, which meanwhile can be used to predict intestinal cancer lesion at the early stage of the cancer. The specific technical solutions are as follows:

In one technical solution, the present application provides a molecular marker for detection of intestinal cancer, said marker is CNPY2 protein isoform 2 having the sequence identified herein as SEQ ID NO:1.

Further, the mRNA sequence of said molecular marker is identified herein as SEQ ID NO: 2.

Further, the cDNA sequence of said molecular marker is identified herein as SEQ ID NO: 3.

In another technical solution, the present application provides a monoclonal antibody, which can specifically bind to CNPY2 protein isoform 2.

In still another technical solution, the present application provides a kit, comprising at least one antibody specifically binding to CNPY2 protein isoform 2.

Preferably, said kit comprises at least two antibodies specifically binding to CNPY2 protein isoform 2.

Further, said kit comprises CNPY2 Protein Capturing Plate, Sample Diluent, Sample Buffer, CNPY2 Standard, Wash Buffer, Enzyme-linked Marker and Substrate Solution.

Preferably, said Enzyme-linked Marker is HRP-labeled Streptavidin, and said Substrate Solution is TMB Substrate Solution.

Preferably, said kit is an ELISA assay kit.

Preferably, said ELISA assay kit is a sandwich ELISA assay kit.

In another technical solution, the present application provides a medication, which may block binding of CNPY2 protein isoform 2 to the receptor thereof, so as to prevent growth of intestinal cancer cells.

Preferably, said medication blocks the binding of CNPY2 protein isoform 2 to the receptor thereof by specifically binding to CNPY2 protein isoform 2.

Preferably, said medication is an antibody specifically binding to CNPY2 protein isoform 2.

In another technical solution, the present application provides a method for the detection, diagnosis and prognosis of intestinal cancer, comprising:

a. determining the concentration of protein isoform 2 from a sample of a suspected individual,
b. comparing the concentration determined in step a with that of CNPY2 protein isoform 2 from a sample of a healthy individual, as compared to the healthy individual, an increase in the concentration of CNPY2 protein isoform 2 indicates a probable presence or a risk of intestinal cancer.

Preferably, said individual is human, said sample is blood, plasma or serum sample.

Preferably, an antibody specifically binding to CNPY2 protein isoform 2 is used to determine the concentration of CNPY2 protein isoform 2.

Preferably, the concentration of CNPY2 protein isoform 2 is determined by ELISA.

Preferably, the determined concentration of CNPY2 protein isoform 2 of the suspected individual is at least 2, 3, 4, 5 or 6 times that of the healthy individual.

Further, the method also determines and compares the concentration of CNPY2 protein isoform 1 of the suspected individual and healthy individual, for combined detection, diagnosis and prognosis of intestinal cancer.

In another technical solution, the present application provides a method for detection and diagnosis of intestinal cancer, comprising:

c. determining mRNA concentration of CNPY2 protein isoform 2 from a sample of a suspected individual,
d. comparing the concentration determined in step a with mRNA concentration of CNPY2 protein isoform 2 from a sample of a healthy individual, wherein, as compared to the healthy individual, an increase in the mRNA concentration of CNPY2 protein isoform 2 indicates a probable presence or a risk of intestinal cancer.

Preferably, the mRNA concentration of CNPY2 protein isoform 2 is determined by PCR.

Preferably, said PCR use a forward primer: 5′-AGACCATTCAGATGGGATCTTTC-3′ (SEQ ID NO:6), and a reverse primer: 5′-TTCATCCAAAGCCAGAGTGAG-3′ (SEQ ID NO:7).

Preferably, the determined mRNA concentration of CNPY2 protein isoform 2 of the suspected individual is at least 2, 3, 4, 5, 6, 7, 8, 9, 10, 11 or 12 times that of the healthy individual.

Further, the method also determines and compares the mRNA concentration of CNPY2 protein isoform 1 of the detected individual and healthy individual, for combined detection, diagnosis and prognosis of intestinal cancer.

In another technical solution, the present application provides a method for the prevention, alleviation or treatment of intestinal cancer, which comprises blocking the binding of CNPY2 protein isoform 2 to the receptor thereof, so as to prevent growth of intestinal cancer cells.

Preferably, the method comprises administration of the above antibody or medication to those with a risk of intestinal cancer or intestinal cancer patients.

In another technical solution, the present application provides a method for the prevention, alleviation or treatment of intestinal cancer, comprising reducing the expression of CNPY2 protein isoform 2.

### DETAILED DESCRIPTION

1. the protein sequence of CNPY2 protein isoform 2
(NM_001190991)
(SEQ ID NO: 1)
MKGWGWLALLLGALLGTAWARRSQDLHCGACRALVDELEWEIAQVDPKKT
IQMGSFRINPDGSQSVVEVTVTVPPNKVAHSGFG

The underlined protein sequence represents a predicted signal peptide which needs to be cut off by signal peptidase to obtain a mature protein. After some posttranslational modifications, the sequence becomes a mature secretory protein and secretes into the blood from cells.

FIG. 1 shows the predicting result of whether the protein sequence of protein isoform of CNPY2 is a secretory protein through a professional website (http://www.cbs.dtu.dk/services/SignalP/). The result demonstrates that the cleavage site on which the signal peptidase cleave is between the 20th and 21th amino acids (AWA-RR), and the whole protein sequence has no hydrophobic trans-membrane domain (also see Table 1).

TABLE 1

| # Measure | Position | Value | Cutoff | Signal peptide? |
|---|---|---|---|---|
| Max. C | 21 | 0.881 | | |
| Max. Y | 21 | 0.918 | | |
| Max. S | 12 | 0.990 | | |
| Max. S | 1-20 | 0.958 | | |
| D | 1-20 | 0.939 | 0.450 | Yes |

Name = Sequence SP = ‘YES’ Cleavage site between pos. 20 and 21: AWA-RR D = 0.939 D-cutoff = 0.450 Networks = SignalP-noTM

2. The mRNA sequence of CNPY2 protein isoform 2
(NM_001190991)
(SEQ ID NO: 2)

| | |
|---|---|
| 1 | attgaaacct ggactgctcg ctggccggca gcgcaccgtt |
| | ttgaaggtcc tagcccacct |
| 61 | gggctggctc acgcgcacga ctagccgctc ccatacagca |
| | cgcccggact ctgtcgtcgc |
| 121 | ttaaggccac tcctattcta cggctgaccc ctggtggtca |
| | cgtggatctg ttcgccacgc |
| 181 | aagtctgggt ccttcggcga ttgaccgggg tccttgctgt |
| | tcgggagcct ctcctaagct |
| 241 | gcctgttcgc gcgagagttt ggaggggcgg gtttggggtc |
| | ggtgtctgat tggggctcgc |
| 301 | accgcagcac gctggagtcc cgcttaggta ccagttagcg |
| | tcaggggagc tgggtcaggc |
| 361 | ggtcgccggg acaccccgtg tgtggcaggc ggcgaagcgc |
| | tctggagaat cccggacagc |
| 421 | cctgctccct gcagccaggt gtagtttcgg gagccactgg |
| | ggccaaagtg agagtccagc |
| 481 | ggtcttccag cgcttgggcc acggcggcgg ccctgggagc |
| | agaggtggag cgaccccatt |
| 541 | acgctaaaga tgaaaggctg gggttggctg gccctgcttc |
| | tgggggccct gctgggaacc |
| 601 | gcctgggctc ggaggagcca ggatctccac tgtggagcat |
| | gcagggctct ggtggatgaa |
| 661 | ctagaatggg aaattgccca ggtggacccc aagaagacca |
| | ttcagatggg atctttccgg |
| 721 | atcaatccag atggcagcca gtcagtggtg gaggtaactg |
| | ttactgttcc cccaaacaaa |
| 781 | gtagctcact ctggctttgg atgaaattcg actgcttaaa |
| | aaggaccttg gtttaataga |
| 841 | aatgaagaaa acagactcag aaaaaagatt tggctctgtc |
| | tcatttggaa gaagctgcag |
| 901 | gcttattccc catgcacttg cttcctggct gcaaacctta |
| | atactttgtt tctgctgtag |
| 961 | aatttgttag caaacaggga gtcctgatca gcacccttct |
| | ccacatccac atgactggtt |
| 1021 | tttaatgtag cactgtggta tacatgcaaa catccgttca |
| | aaatctgagt cggagctaaa |
| 1081 | aataaaaaat gaaaaaacag aaataagaat aaaaggtctt |
| | atcctcataa aaaaaaaaaa |
| 1141 | aaaaaaaaa |

The underlined sequence is cDNA (SEQ ID NO:3) that can be translated into the protein, wherein the initiation codon ATG is located in 550-552; the termination codon TGA is located in 802-804.

3. the production of CNPY2 isoform 2 monoclonal antibody and sandwich ELISA kit

The inventors of the present application devote their attention to the possibility of using new gene CNPY2 isoform 2 as an early molecular marker for diagnosis of intestinal cancer. For the first time, they developed unique five mouse monoclonal antibodies in the world against this new CNPY2 isoform 2 in about 10 months.

The producing process of the five monoclonal antibodies starts from cloning the full-length cDNA of human CNPY2 isoform 2 (not comprising the anterior 20 amino acids as they form a signal peptide) into an expression vector pET30a, with 6×His-tag in the tail, and then expressing the protein in E. Coli BL21 (DE3). For its insolubility after expression, the protein can be purified from inclusion bodies.

FIG. 2 is the SDS-PAGE analysis diagram of CNPY2 isoform 2, wherein, lane 1: BSA (5.00 μg); lane 2: CNPY2 (4.60 μg); FIG. 3 is the Western blot analysis diagram of CNPY2 isoform 2, using anti-His antibody, wherein, lane 3: CNPY2 (275 μg).

### Derived Protein Sequence:

TABLE 2

| Theoretical Isoelectric Point | Theoretical Molecular Weight |
|---|---|
| 7.27 | 7960.0 Da |

MRRSQDLHCGACRALVDELEWEIAQVDPKKTIQMGSFRINPDGSQSVVEV
TVTVPPNKVAHSGFGHHHHHH

The underlined sequence is obtained by purifying the recombinant protein of CNPY2 isoform 2 after its expression in E. Coli. The sequence does not have the anterior 20 amino acids (signal peptide), while in the tail 6 His-tags are added.

Five monoclonal antibodies are finally selected by immunizing mice with the recombinant protein of CNPY2 isoform 2, extracting splenocytes, fusing the cells with myeloma cells to produce hybridoma cell lines, and then performing ELISA screening through two artificial protein segments.

The first protein segment is CVEVTVTVPPNKVAHSGFG (SEQ ID NO:4), the protein sequence of C-terminus in the tail of new gene CNPY2 isoform 2, which is a unique protein sequence totally different from that of new gene CNPY2 isoform 1 or other unrelated genes.

The second protein segment is CTIQMGSFRINPDGSQSV (SEQ ID NO:5), the protein sequence of N-terminus in the head of new gene CNPY2 isoform 2 with the signal peptide cut off. This protein sequence is exactly the same in CNPY2 isoform 1 and CNPY2 isoform 2.

The first batch of sandwich ELISA assay kits are produced through repeated pairing test and adjustment, wherein the monoclonal antibody 13G11B9 is selected as the best pair for coating (concentration of 2 μg/ml), and the biotin-labeled 2B11D11 for detection (concentration of 1 μg/ml). This 60 sandwich ELISA assay kits have been packed and adjusted with the sensitivity up to 10 pg/ml serum or plasma, which can be used for serological detection of intestinal cancer patients.

4. CNPY2 protein isoform 2 is a secretory protein

The present inventors then clone the cDNA sequence of human CNPY2 isoform 2 into the mammalian expression plasmid vector pTT5 (see FIG. 4), followed by transfecting it into HEK293 and CHO cell lines for 1-3-5 days, and Western-blotting is used for detecting the protein in cell supernatant with a self-made monoclonal antibody. 3-5 days after transfection, the expression of the protein is detected. As can be seen, the CNPY2 isoform 2 protein is about 10 KDa in reductive SDS-PAGE electrophoresis gel; while in non-reductive SDS-PAGE electrophoresis gel, the protein forms a dimer which is about 20 KDa (see FIG. 5). CNPY2 isoform 2 has a bit higher expression in the supernatant of HEK293 cells than that of CHO cells, which firstly demonstrates that CNPY2 isoform 2 is a secretory protein, that is, firstly demonstrating intestinal cancer can be diagnosed by determining the content of this protein in blood.

5. The expression of CNPY2 isoform 2 mRNA in human intestinal cancer tissues is 12 times higher than that in adjacent tissues

Comparing cancer tissues of 60 intestinal cancer patients with their adjacent normal tissues frozen in the laboratory of Professor Wan Desen from the Colorectal Department of Sun Yat-Sen University Cancer Hospital using Real-time PCR, the present inventors find that the mRNA level of new gene CNPY2 isoform 2 in intestinal cancer tissues is 12 times higher than that in adjacent normal tissues on average, P<0.0001 (see FIG. 6). Also see Table 10, the raw data of PCR.

(SEQ ID NO: 6)
The forward primer: 5′-AGACCATTCAGATGGGATCTTTC-3′
(SEQ ID NO: 7)
The reverse primer: 5′-TTCATCCAAAGCCAGAGTGAG-3′

PCR product=111 bp

DNase is used in the whole RNA to prevent genomic DNA contamination.

SYBR Real-time PCR condition: 95° C., 15 min; 95° C., 15 secs; 60° C., 1 min; 40 cycles.

6. The expression of CNPY2 isoform 2 protein in human intestinal cancer tissues is 6 times higher than that in adjacent tissues.

The present inventors use the slices of biopsy intestinal cancer tissues frozen in the laboratory of Pro. Wan for immunohistochemistry staining, comparing the expression of protein of new gene CNPY2 isoform 2 in cancer tissues and adjacent normal tissues. A monoclonal antibody (clone 20E12B5) selected from 5 monoclonal antibodies in the preliminary experiment has the best staining effect. The protein sequence of antigenic determinant (epitope) corresponding to this monoclonal antibody (clone 20E12B5) is unique for new gene CNPY2 isoform 2. Thus, this antibody will not have cross reaction with the protein of CNPY2 isoform 1 or other unrelated proteins. See FIG. 7: the immunohistochemistry staining result by the monoclonal antibody (clone 20E12B5), wherein dark brown represents intestinal cancer tissue cells.

Finally, this monoclonal antibody (clone 20E12B5) is used for immunohistochemistry of large sample. After scoring (12 points scoring criteria of standard pathology) by doctors from the Department of Pathology of Sun Yat-sen University Cancer Hospital, the result of large sample experiment using tissue slices from more than three hundred of intestinal cancer patients at different clinical stages is inspiring (see Table 11), which also firstly proves that the expression of the protein of this new gene CNPY2 isoform 2 does exist objectively. Furthermore, the average expression level of this protein in carcinoma tissues of intestinal cancer patients at stages 1-4 is over 6 times higher than that in adjacent normal tissues, P<0.0001.

(1) The expression of CNPY2 isoform 1 in intestinal cancer tissues at stages 1, 2 and 4 and normal tissues (see FIG. 8).

The experiment shows that, the average expression level of CNPY2 isoform 1 in intestinal cancer tissue of stages 1, 2 and 4 is 6 times higher than that in adjacent normal tissues (normal tissues: N=275, mean±SEM=1.132±0.08138; tumor tissues: N=327, mean±SEM=7.315±0.1690; the difference between mean values: 6.183±0.1989; P<0.0001).

(2) The experiment results of CNPY2 isoform 2 in intestinal cancer at stage 1 The immunohistochemical score of CNPY2 isoform 2 in intestinal cancer at stage 1 (see FIG. 9, normal tissues: N=51, mean±SEM=1.340±0.1762; tumor tissues: N=62, mean±SEM=8.118±0.3248; the difference between mean values: 6.778±0.3924; P<0.0001).

The immunohistochemical staining of CNPY2 isoform 2 demonstrating that, the score of the protein in intestinal cancer tissues at stage 1 is 6.8 times higher than that of normal tissues on average (see FIG. 10).

(3) The experiment results of CNPY2 isoform 2 in intestinal cancer at stage 2

The immunohistochemical score of CNPY2 isoform 2 in intestinal cancer at stage 2 (see FIG. 11, normal tissues: N=82, mean±SEM=1.167±0.1592; tumor tissues: N=104, mean±SEM=7.547±0.3025; the difference between mean values: 6.379±0.3689; P<0.0001).

The immunohistochemical staining of CNPY2 isoform 2 demonstrating that, the score of the protein in intestinal cancer tissues at stage 2 is 6.4 times higher than that in normal tissues on average (see FIG. 12).

(4) The experiment results of CNPY2 isoform 2 in intestinal cancer at stage 4 The immunohistochemical score of CNPY2 isoform 2 in intestinal cancer at stage 4 (see FIG. 13, normal tissues: N=142, mean±SEM=1.038±0.1113; tumor tissues: N=161, mean±SEM=6.857±0.2483; the difference between mean values: 5.819±0.2843; P<0.0001).

The immunohistochemical staining of CNPY2 isoform 2 demonstrating that, the score of the protein in intestinal cancer tissues at stage 4 is 5.8 times higher than that in normal tissues on average (see FIG. 14).

After wide and deep research, the inventors draw the following main conclusions:

1. The cDNA of CNPY2 isoform 2 (Genbank accession number NM_001190991) totally has 255 base pairs, and the protein product translated has 84 amino acids, wherein the anterior 20 amino acids is a signal peptide. After synthesis of protein, this signal peptide will be cut off in the endoplasmic reticulum ER by signal peptidase before becoming a secretory protein, and the molecular weight of the finally secreted single-strand protein is about 10 KDa. Naturally, the protein will form a dimer, with molecular weight of about 20 KDa. This protein can be detected in the supernatant of transfected HEK293 and CHO cell lines by conventional western blotting using monoclonal antibodies. Therefore, it is able to be detected in serum or plasma of human or other mammals using ELISA kit (e.g. sandwich ELISA kit).

2. The expression of CNPY2 isoform 2 mRNA in cancer tissues of intestinal cancer patients is on average about 12 times higher than that in adjacent normal tissues of the same patients, N=60, P<0.0001.

3. The expression of CNPY2 isoform 2 protein in cancer tissues of intestinal cancer patients at clinical stages 1, 2 and 4 is on average about 6 times higher than that in adjacent normal tissues of the same patients, N=˜300, P<0.0001.

4. CNPY2 isoform2 protein can be used as a molecular marker for intestinal cancer in human and other mammals

5. As a secretory ligand, CNPY2 isoform2 protein is supposed to bind to a receptor on the surface of cell membrane, start cell signal delivering, activate some downstream kinases involved in cell cycle and accelerate cancer cell division. A humanized blocking monoclonal antibody or small molecular compound may prevent cancer cell growth if it is able to block the binding of CNPY2 isoform 2 (as a ligand) to the receptor thereof, so as to prevent and treat intestinal cancer.

### DESCRIPTION OF THE DRAWINGS

The above and other objects, advantages and features of the present invention will become more apparent to those skilled in the art according to the following detailed descriptions of specific examples of the invention in combination with the drawings.

FIG. 1. SignalP-4.1 prediction (euk network ssequence) graph The graph shows that, the cleavage site on which the signal peptidase cut off is between the 20th and 21th amino acids (AWA-RR), and the whole protein sequence has no hydrophobic transmembrane domain.

FIG. 2. SDS-PAGE analysis graph of CNPY2 isoform 2 Lane 1: BSA (5.00 μg); Lane 2: CNPY2 (4.60 μg). FIG. 3. Western blot analysis graph of CNPY2 isoform 2, using anti-His antibody. Lane 3: CNPY2 (275 μg).

FIG. 4. expression plasmid vector pTT5 The cDNA sequence of human CNPY2 isoform 2 is cloned into mammalian expression plasmid vector pTT5.

FIG. 5. The expression and detection of CNPY2 protein isoform 2 in transfected cell lines. the CNPY2 isoform 2 protein is about 10 KDa in reductive SDS-PAGE electrophoresis gel; while in non-reductive SDS-PAGE electrophoresis gel, the protein forms a dimer which is about 20 KDa

FIG. 6. The expression of CNPY2 isoform 2 mRNA The blank column represents the expression level of CNPY2 isoform 2mRNA in adjacent normal tissues, and the solid column represents the expression level of CNPY2 isoform 2mRNA in intestinal cancer tissues. The results show that, the level of CNPY2 isoform 2 mRNA in intestinal cancer tissues is on average 12 times higher than that in adjacent normal tissues.

FIG. 7. The results of immunohistochemical staining by monoclonal antibody (clone 20E12B5) of CNPY2 isoform 2. Dark brown in the graph represents intestinal cancer tissue cells.

FIG. 8. The immunohistochemical score of CNPY2 isoform 1 in intestinal cancer tissues at stages 1, 2 and 4 and normal tissues. Normal tissues: N=275, mean±SEM=1.132±0.08138; tumor tissues: N=327, mean±SEM=7.315±0.1690; the difference between mean values: 6.183±0.1989; P<0.0001.

FIG. 9. The immunohistochemical score of CNPY2 isoform 2 in intestinal cancer at stage 1. Normal tissues: N=51, mean±SEM=1.340±0.1762; tumor tissues: N=62, mean±SEM=8.118±0.3248; the difference between mean values: 6.778±0.3924; P<0.0001.

FIG. 10. The immunohistochemical staining of CNPY2 isoform 2 in intestinal cancer at stage 1. The graph shows that, the score of the protein in intestinal cancer tissues at stage 1 is 6.8 times higher than that of normal tissues on average.

FIG. 11. The immunohistochemical score of CNPY2 isoform 2 in intestinal cancer at stage 2. Normal tissues: N=82, mean±SEM=1.167±0.1592; tumor tissues: N=104, mean±SEM=7.547±0.3025; the difference between mean values: 6.379±0.3689; P<0.0001.

FIG. 12. The immunohistochemical staining of CNPY2 isoform 2 in intestinal cancer at stage 2. The graph shows that, the score of the protein in intestinal cancer tissues at stage 2 is 6.4 times higher than that in normal tissues on average.

FIG. 13. The immunohistochemical score of CNPY2 isoform 2 in intestinal cancer at stage 4. normal tissues: N=142, mean±SEM=1.038±0.1113; tumor tissues: N=161, mean±SEM=6.857±0.2483; the difference between mean values: 5.819±0.2843; P<0.0001.

FIG. 14. The immunohistochemical staining of CNPY2 isoform 2 in intestinal cancer at stage 4. The graph shows that, the score of the protein in intestinal cancer tissues at stage 4 is 5.8 times higher than that in normal tissues on average.

FIG. 15. The principle of CNPY2 ELISA assay kit.

FIG. 16. The detection procedure graph for detecting CNPY2 protein in samples.

FIG. 17. CNPY2 standard curve.

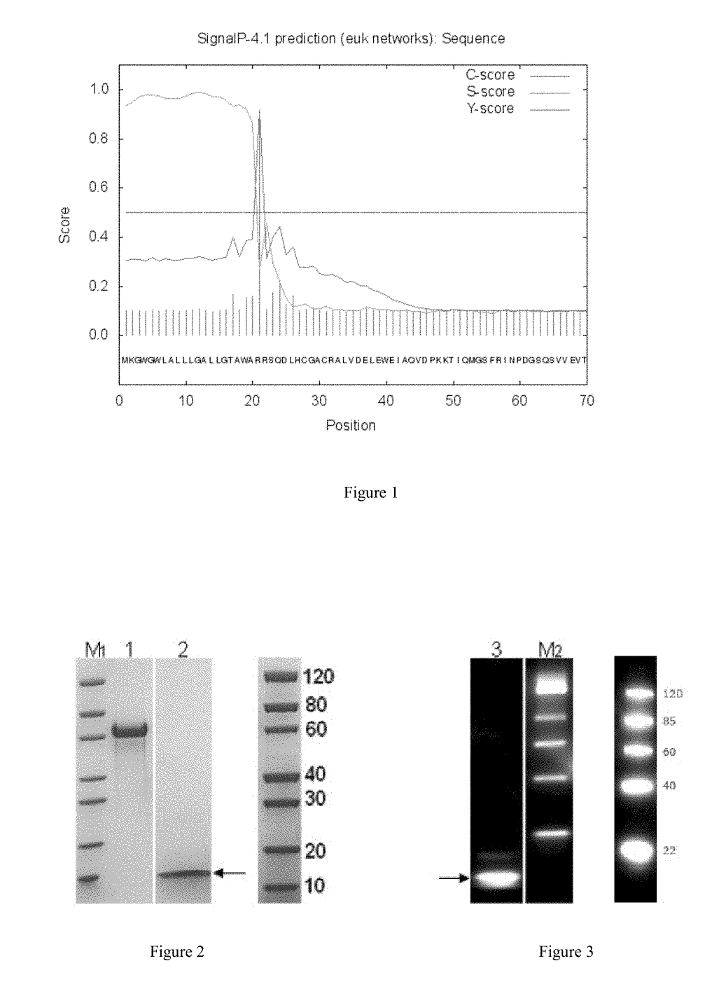

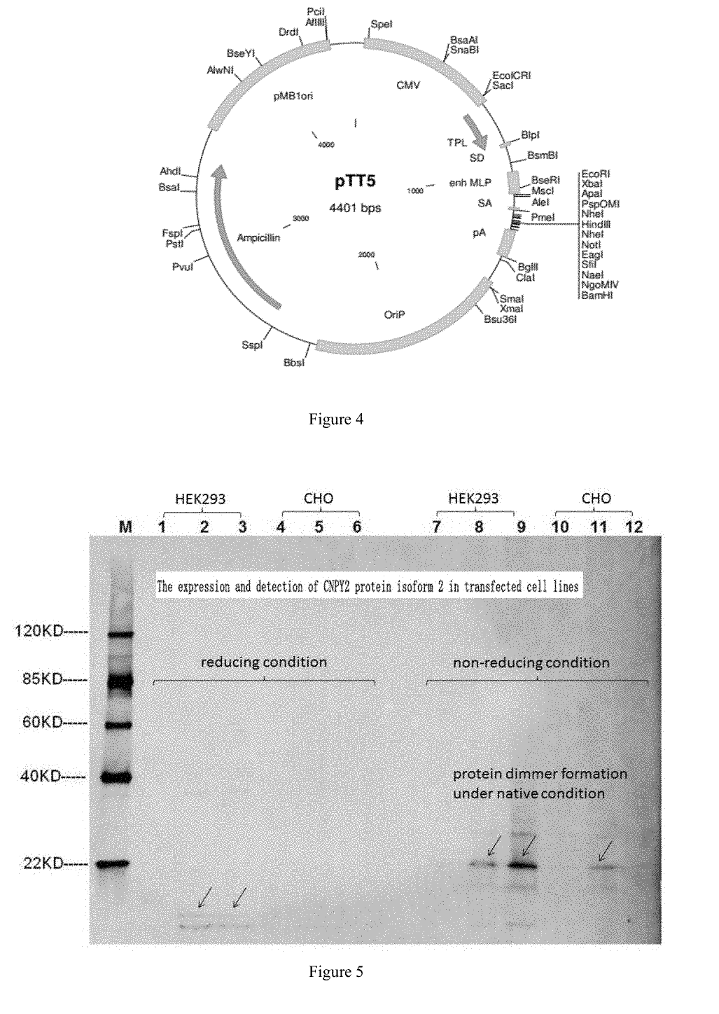

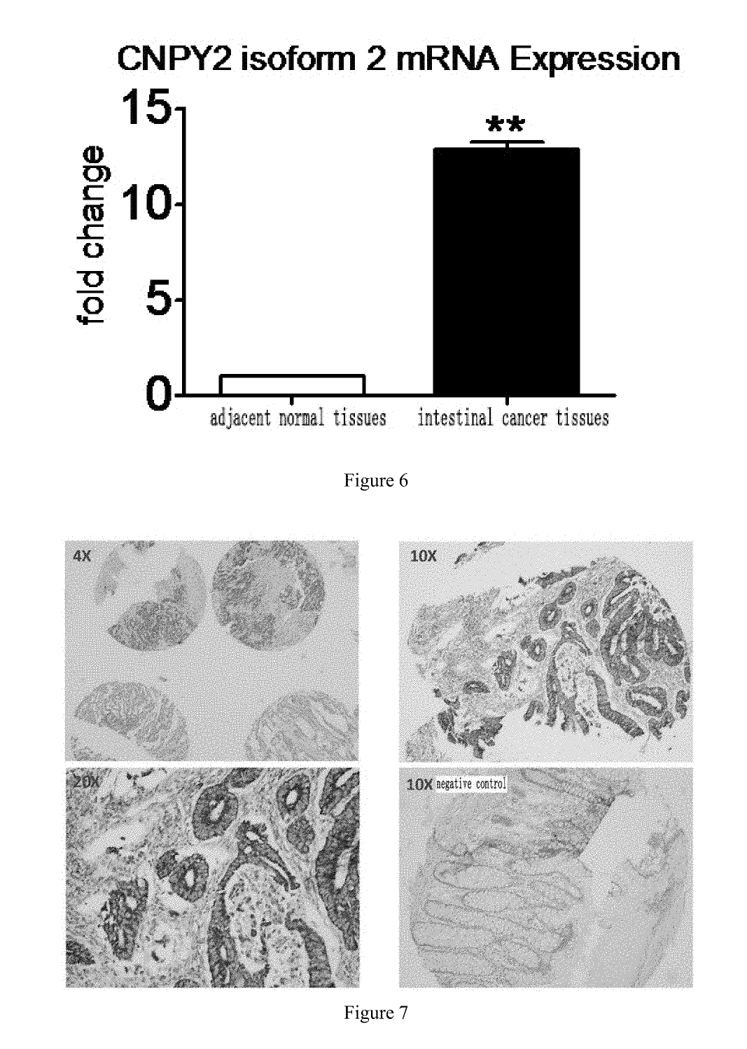

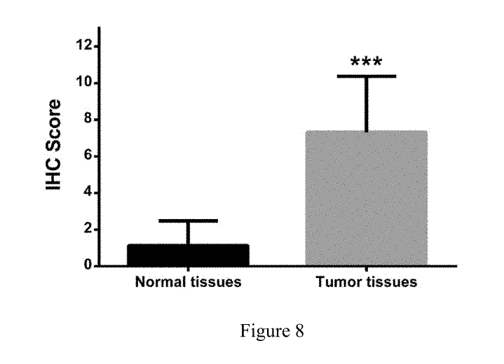

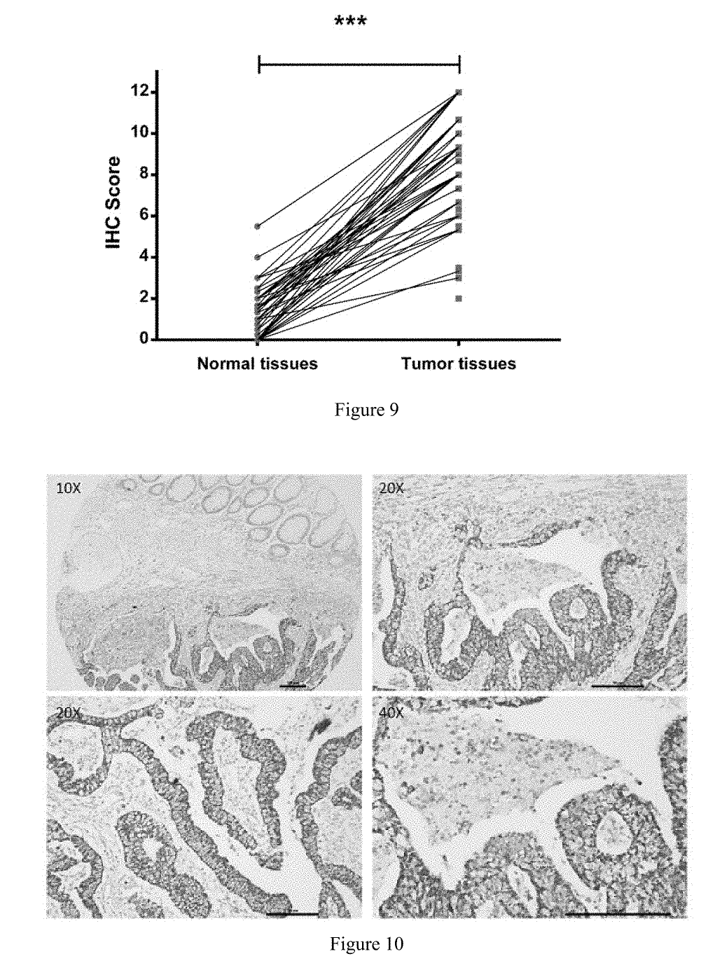

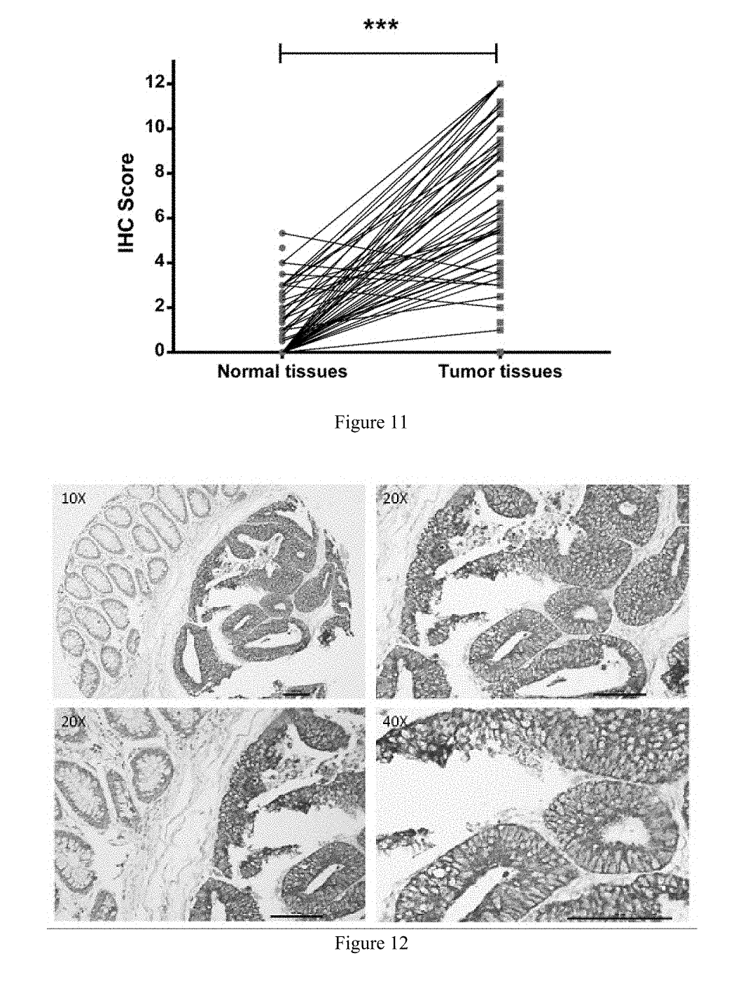

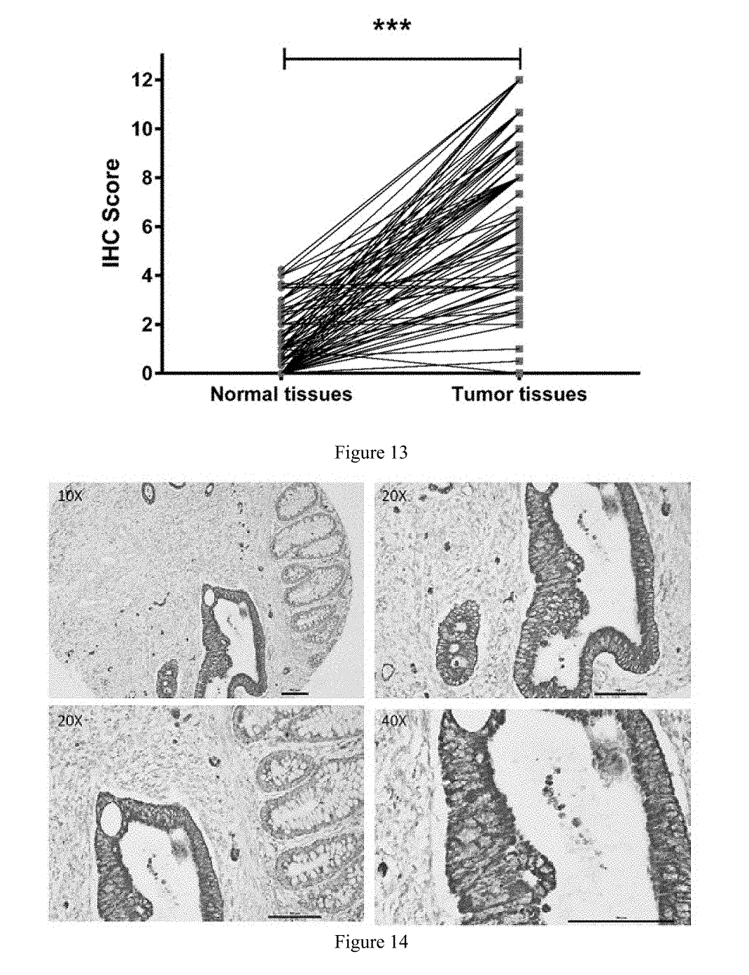

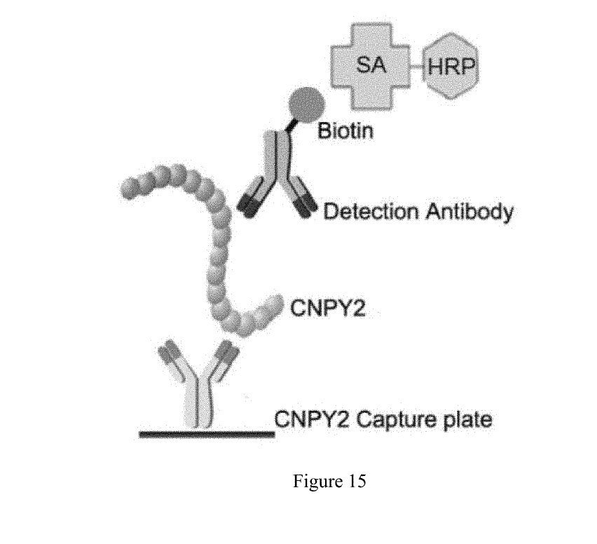

### DESCRIPTION OF THE PREFERRED EMBODIMENTS

The present invention is further illustrated by specific examples, and it should be understood that the following specific embodiments are only intended to illustrate the present invention and do not limit the content of the invention.

The materials and equipments used in the examples are well known to those skilled in the art and are commercially available or readily available or prepared.

### Example 1. Monoclonal Antibody (Mab) Development

1. Project Objective

To develop mouse monoclonal antibody (Mab) specific to CNPY2 protein that could be paired in sandwich ELISA application.

2. Materials

CNPY2 protein and peptide cTIQMGSFRINPDGSQSVVEVTVTVPPNKVAHSGFG.

3. Project Procedures

Milestone 1, Animal immunization

6 animals (3 Balb/c mice+3 C57 mice) will be immunized with CNPY2 protein as in Table 3.

TABLE 3
Immunization Schedule

| Procedure | Schedule | Dosage/Route |
|---|---|---|
| Pre-Immune Bleed | T = −4 days | |
| Primary Immunization | T = 0 days | 100-50 μg/animal, s.c./i.p. |
| 1^{st}Boost | T = 14 days | 50-25 μg/animal, s.c./i.p. |
| Test Bleed 1 | T = 21 days | |
| 2^{nd}Boost | T = 28 days | 25-50 μg/animal, s.c./i.p. |
| Test Bleed 2 | T = 35 days | |
| Final Boost | T = 56 + 7 days | 25-50 μg/animal, i.p./i.v. |

| | |
|---|---|
| Cell Fusion | T = Final boost + 4 days |

1) Test bleed: Immune response tests by ELISA with immunized sera will be carried out 7 days after each boost immunization. Test bleed by ELISA with target protein and peptide.

2) Deliver antiserum to the client for in-house test if requested.

3) Maintain the immunized animals until completion of project.

Decision gate: After Milestone 1, if the animals produce good immune responses against the immunogen, the projects will be continued, and preferred animal can be selected for cell fusion. If the immune responses are not qualified for cell fusion, solutions will be discussed on for improvement.

Deliverables: Antiserum.

Timeline: 8-10 weeks.

Milestone 2, Cell fusion and screening

1) Animal selection: according to the test bleed result, top two animals with the best immune responses against the target peptide will be selected for cell fusion. Fusions can be staggered.

2) Cell fusion and clone plating: 2 fusions will be performed by electrofusion. A fusion efficiency around 1 hybridoma/5000 B cells is observed. Based on such experience, with an average of 1×10⁸ B cells from each spleen of immunized mouse, the anticipated recovery of hybridoma clones is about 2×10⁴. All fused cells from each cell fusion will be plated into 10 96-well plates.

3) Primary positive screening: screen the supernatants by ELISA with target protein for positive screening.

4) Confirmatory screening: confirmatory screening is carried out by testing the supernatants of all positive clones identified in primary screening by indirect ELISA against target protein and peptide.

5) Clone selection and frozen: up to 10 positive parental clones, which are specific to the CNPY2 protein, are expected. Expand all positive clones into 24-well plates. Collect 2 ml of supernatant (conditioned media) for each clone and freeze down the cells. Samples of hybridoma culture supernatant (2 ml per clone) will be sent for in-house testing if requested. All specific positive clones will be frozen down to avoid clone lost.

Decision gate: specific positive clones are selected to be performed following subcloning.

Deliverables: samples of parental clone culture supernatant (2 ml)

Timeline: 4-6 weeks

Milestone 3, Subcloning, expansion and cryopreservation

1) Subcloning selection: up to 5 positive primary clones selected will be subcloned by limiting dilution to ensure the subclones are derived from a single parental cell. The clones will be carried for a maximum of 3 generations. It is anticipated up to 4 primary clones (about 80% successful rate) will survive in subcloning stage and grow stably (If the positive clones do not meet the specificity requirement, additional parental clones may be selected to repeat the subcloning).

2) Subcloning screening: subcloning will be screened by ELISA.

3) Monoclone cryopreservation: two stable subclonal cell lines of each primary clone will be chosen for cryopreservation based on confirmed antigen-recognition and normal doubling time.

4) Isotype identification for all the subcloned cell lines. All the obtained clones will be preserved. The IgG isotype is preferred.

Decision gate: positive clones will be selected to be produced for antibody production, conjugation and mab-pab pairing.

Deliverables: 2 vials of frozen cells and 5 ml supernatant for each subcloned monoclonal cell line.

Timeline: 4-6 weeks.

Milestone 4, Monoclonal antibody production

1) Antibody production: Produce antibody with roller bottle culture or ascites production for each selected cell lines (up to 5 cell lines), purify the produced antibodies by protein A/G affinity column. 2-5 mg purified antibody will be produced for the selected clones.

2) Antibody validation: Test the produced antibodies for purity by SDS-PAGE, concentration by OD280 nm, and reactivity by ELISA.

Deliverables: 2-5 mg purified antibody will be produced for each selected clone, purity >90%, concentration>0.4 mg/ml.

Timeline: 4-6 weeks.

As a result, five unique mouse monoclonal antibodies in the world are obtained against the new gene CNPY2 isoform 2. These five monoclonal antibodies are: 13G11B9, 14G9B9, 20E12B5, 2B11D11 and 8D1F8.

Among the antibodies, the epitope corresponding to monoclonal 13G11B9, 14G9B9 and 20E12B5 is CVEVTVTVPPNKVAHSGFG (SEQ ID NO:4), which is unique to CNPY2 isoform 2. The epitope corresponding to monoclonal 2B11D11 and 8D1F8 is CTIQMGSFRINPDGSQSV (SEQ ID NO:5).

In the experiment, monoclonal 20E12B5 has the best immunostaining effect.

### Example 2. ELISA Development

1. Project Description

To develop an immunoassay kit which minimum detectable dose (MDD) is expected to reach 100 pg/ml.

2. Material

Test sample, 5-10 positive samples and 5-10 negative samples. At least 1 ml per sample is required. (Used in milestone 3)

3. Project procedure

Milestone 1, Prove of concept (POC)

1) HRP or Biotin conjugation for the selected detection antibodies, up to 5 antibodies.

2) Identify best pairs and evaluate the performance in sensitivity and specificity.

3) Select the best pair for sandwich ELISA.

4) Study the feasibility of the best pair with sandwich ELISA.

5) Evaluate the consistent data with the inventor.

Decision gate: At the conclusion of milestone 2, estimate the sensitivity of the antibody pair, the inventor can select if the project should be go on or terminated.

Timeline: 1-2 weeks

Deliverables: Milestone report.

Milestone 2, Assay development

1) Set up a most suitable immunoassay with the selected antibody format based on milestone 2.

2) Select the most suitable assay format and optimize the assay conditions and parameters, e.g. concentration of coating antibody and detection antibody, blocking buffer, blocking time, reaction time and temperature, working buffer. etc.

3) Prepare and validate the assay standards, determine the assay sensitivity and other performances.

4) Sensitivity: The minimum detectable dose (MDD) was determined by adding three standard deviations to the mean relative light unit (RLU) of twenty zero standard replicates and calculating the corresponding concentration.

Decision gate: At the conclusion of milestone 2, estimate the sensitivity of the antibody pair, the inventor can select if the project should be go on or terminated.

Timeline: 1-weeks

Deliverables: (1) milestone report; (2) the optimized parameters of assay.

Milestone 3, Assay validation

1) Pilot manufacture of assay kits (10 kits) for assay validation.

2) Prepare the golden standards using the validated calibrators or validated method.

3) Validate the stability, accuracy and variation of the assay kits, the criterion of the assay kit is:

1). Intra-assay Precision (Precision within an assay)<5%.

Three samples of known concentration were tested eight times on one plate to assess intra assay.

2). Inter-assay Precision (Precision between assays)<10%

Three samples of known concentration were tested in four separate assays to assess inter assay.

3). Recovery range: 100±15%

The recovery of peptide spiked to three different levels in samples throughout the range of the Assay in various matrices was evaluated.

4) Validate the sensitivity and specificity with clinical or field samples.

Decision gate: At the conclusion of milestone 3, the inventor will select if following Assay manufacturing is needed.

Timeline: 3-4 weeks, more time for more samples.

Deliverables:

1) 5 ELISA kit; 2) Milestone report.

Milestone 4, Assay manufacturing

Manufacture 45 immunoassay kits in 96-tests format.

Timeline: 2-3 weeks, more time for more samples.

Deliverables:

1) 45 ELISA kit; 2) Milestone report.

The sandwich ELISA assay kits are produced after repeated pairing test and adjustment, wherein the monoclonal antibody 13G11B9 is selected as the best pair for coating (concentration of 2 μg/ml), and the biotin-labeled 2B11D11 for detection (concentration of 1 μg/ml).

### Example 3. Kit Development and Detection

1. Kit Description

The kit detects CNPY2 protein in the sample using solid phase double antibody sandwich enzyme-linked immunoassay. Meanwhile, monoclonal antibody also can accurately detect CNPY2 protein, which is available by the skilled in the art. Anti-CNPY2 protein monoclonal antibody was used to coat microplate and produce solid-phase antibody, and standard and sample to be detected were added into the corresponding microplate coated with monoclonal antibody, wherein the CNPY2 protein may bind to the corresponding anti-CNPY2 protein antibody in the plate well to form an antigen-antibody complex; then, biotin-labeled anti-CNPY2 protein monoclonal antibody was added; further, Streptavidin-HRP conjugate (SA-HRP) was added to form anti-CNPY2-antibody-biotin-SA-HRP complex, and TMB substrate was added for developing after washing; TMB turns to blue with HRP and finally converts to yellow with acid, where the depth of color is positively correlated with the content of CNPY2 protein in the sample.

2. Kit Components

The kit provides all the reagents requested for detection of samples, see Table 4, the reagents are enough for detection on one plate.

TABLE 4

| Components | Quantity | | Part No. |
|---|---|---|---|
| CNPY2Protein Capture | 1 plate (8wells × | | 688699-80 |
| Plate | 12strips) | | |
| 5 × Sample Diluent | 20 | ml | 688699-60 |
| Sample Buffer | 15 | ml | 688699-90 |
| Detection antibody stock | 500 | μl | 688699-20 |
| Streptavidin-HRP | 500 | μl | 688699-30 |
| CNPY2 Standard | 2 | vials | 688699-10 |
| 20 × Wash Buffer | 30 | ml | 688699-70 |
| Substrate Solution A | 12 | ml | 688699-40 |
| Substrate Solution B | 12 | ml | 688699-41 |
| Stop Solution | 6 | ml | 688699-50 |

3. Preservation

CNPY2 protein standard in the kit should be stored at −20° C. or −80° C., other parts can be stably preserved for 1 year at 2-8° C.

4. Materials Requested

The following are materials and devices requested in the experiment which are not available in the kit:

Microplate reader for detecting 450 nm absorbance;

Automatic plate washer;

Deionized water or double distilled water;

Cylinder;

1000 mL beaker;

Different sizes of EP tubes;

Different sizes of precision micropipettes, multichannel micropipettes and tips;

Tower paper;

Timer;

−20° C. refrigerator, 4° C. incubator, 25° C. incubator (if the room temperature cannot reach 25±2° C., 25° C. incubator is recommended) and 37° C. incubator;

centrifuge.

5. Experiment procedure

(1). Reagent preparation

1× Wash Buffer

Dilute 20× Wash Solution with double-distilled or deionized water by 1:20. For example, dilute 10 ml of 20× Washing Solution with 190 ml of double-distilled or deionized water to make 200 ml of Wash Solution, with stored at 2-8° C.

Note: If any precipitate forms in the 20× Wash Solution during storage, incubate the bottle in water bath until all the precipitate disappears.

1× Sample Dilution Solution

Dilute 5× Sample Dilution Solution with double-distilled or deionized water by 1:5. For example, dilute 20 ml of 5× Sample Dilution Solution with 80 ml of double-distilled or deionized water to make 100 ml of 1× Sample Dilution Solution, with stored at 2-8° C.

CNPY2 Standard Solution

CNPY2 Standard is reconstituted by adding 450 μl of deionized water (or double-distill water). This reconstitution provides the CNPY2 stock solution with the CNPY2 concentration of 32 ng/ml.

Note: The reconstitution should be used immediately, it cannot be used for the next time.

With reference to the following table 5 dilution steps to formulate the standard curve 640 pg/mL, 320 pg/mL, 160 pg/mL, 80p g/mL, 40 pg/mL, 20p g/mL, 10 pg/mL and 0 pg/mL.

TABLE 5

| | | CNPY2 preparing |
|---|---|---|
| Transfer | Add to | concentration |
| 20 μl of 32 pg/mL standard | 980 μl × Sample | 640 pg/mL |
| | Diluent, | |
| | mixing | |
| 300 μl of 640 pg/mL standard | 300 μl × Sample | 320 pg/mL |
| | Diluent, | |
| | mixing | |
| 300 μl of 320 pg/mL standard | 300 μl × Sample | 160 pg/mL |
| | Diluent, | |
| | mixing | |
| 300 μl of 160 pg/mL standard | 300 μl × Sample | 80 pg/mL |
| | Diluent, | |
| | mixing | |
| 300 μl of 80 pg/mL standard | 300 μl × Sample | 40 pg/mL |
| | Diluent, | |
| | mixing | |
| 300 μl of 40 pg/mL standard | 300 μl × Sample | 20 pg/mL |
| | Diluent, | |
| | mixing | |
| 300 μl of 20 pg/mL standard | 300 μl × Sample | 10 pg/mL |
| | Diluent, | |
| | mixing | |
| 300 μl of Sample Diluent | 300 μl × Sample | 0 pg/mL |
| | Diluent, | |
| | mixing | |

Detection Antibody Working Solution

Detection antibody concentrate solution was diluted to working concentration with 1× Sample Diluent at a ratio of 1:50, e.g., adding 4.9 ml 1× Sample Diluent into 100 ul detection antibody concentrate solution to prepare detection antibody working solution.

HRP-Labeled Streptavidin Working Solution

HRP-labeled Streptavidin was diluted to working concentration with 1× Sample Diluentat at ratio of 1:50, e.g., adding 4.9 ml 1× Sample Diluent into 100 ul detection antibody concentrate solution to prepare HRP-labeled Streptavidin.

Substrate Solution

Substrate Solution A and Substrate Solution B were mixed at a ratio of 1:1, e.g., to prepare 5 ml Substrate Solution, 2.5 ml Substrate Solution A and 2.5 ml Substrate Solution B were added into centrifuge tube followed by slightly mixing. (Note, Substrate Solution should be used immediately at development step)

(2). Sample Preparation

Pay attention to the following points when preparing the samples:

1.pH of the test samples should be adjusted to Neutral, insoluble substance should be removed by centrifuging or filtering by centrifugation or filtration.

2. Through a pre-experiment to determine the best detection diluted factors of the samples. For example, the samples should be diluted with sample dilution by 1:2, 1:5, 1:10, 1:20.

Standard and sample dilution design was shown in Table 6.

TABLE 6

| | Standard (pg/mL) | | Diluted samples | | | | | | | | | |
|---|---|---|---|---|---|---|---|---|---|---|---|---|
| | Repetition 1 | Repetition 2 | 3 | 4 | 5 | 6 | 7 | 8 | 9 | 10 | 11 | 12 |
| A | 640 | 640 | 1:2 | 1:2 | | | | | | | | |
| B | 320 | 320 | 1:5 | 1:5 | | | | | | | | |
| C | 160 | 160 | 1:10 | 1:10 | | | | | | | | |
| D | 80 | 80 | 1:20 | 1:20 | | | | | | | | |
| E | 40 | 40 | | | | | | | | | | |
| F | 20 | 20 | | | | | | | | | | |
| G | 10 | 10 | | | | | | | | | | |
| H | 0 | 0 | | | | | | | | | | |

(3). Capture Plate preparation

It is recommended that all standards and samples are prepared in duplicate.

1). Reagents and plates (unfolded) should be fully equilibrated to room temperature (20-25° C.) before performing a test. 2.

2). Determine the number of microwell strips required in the test, remove the remaining strips and repack them in the aluminum foil bag, being sealed and stored at 2-8° C. (opened strips should be used in two weeks).

3). Ensure the strips are stably snapped on the frame.

(4). Detection Procedure

Slide the finger across the frame and strips when covering the plate with covering film to ensure the wells are completely sealed.

Measuring reaction time by the timer.

Wash the plate with the fully automatic plate washer or multi-channel micropipette.

Standards and Samples Incubation

1). Pipette 100 μl of Sample buffer to each well of the plate at the same time add 100 μl diluted CNPY2 and Samples to different wells.

2). Cover the plate with adhesive plate cover and incubate at 4° C. for 90 min.

3). Remove the adhesive plate cover and aspirate the solution from the wells.

4). Wash each well of the plate with 260 μl of prepared Wash Solution for four times.

5). Invert the plate and pound it vigorously on clean paper towels to remove excess liquid in each well.

Detection Antibody Working Solution Incubation

6). Pipette 200 μl of detection antibody working solution to each well.

7). Cover the plate with adhesive plate cover and incubate the plate at 4° C. for an hour.

8). Remove the adhesive plate cover and aspirate the solution from the wells.

9). Wash the plate as the step 4.

10). Invert the plate and pound it vigorously on clean paper towels to remove excess liquid in each well.

Streptavid-HRP Working Solution

11). Pipette 200 μl of Streptavid-HRP working solution to each well.

12). Cover the plate with adhesive plate cover and incubate the plate at 37° C. for 10 minutes.

13). Remove the adhesive plate cover and aspirate the solution from the wells.

14). Wash the plate as the step 4.

15). Invert the plate and pound it vigorously on clean paper towels to remove excess liquid in each well.

Substrate Reaction and Absorbance Measurement

16). Pipette 200 μl substrate to each well.

17). Cover the plate with adhesive plate cover and incubate at 25° C. for 15 minutes (starting from adding Substrate Solution into the first well) in darkness environment.

18). Pipette 50 μl of Stop Solution to each well to stop the reaction.

19). Read the plate at 450 nm with a Microplate reader to obtain absorbance values.

Note: The actual development reaction time depends on the temperature, and the ultimatum reaction temperature is 25° C. If temperature is low, reaction time should be extended.

6. Detection Procedure Graph

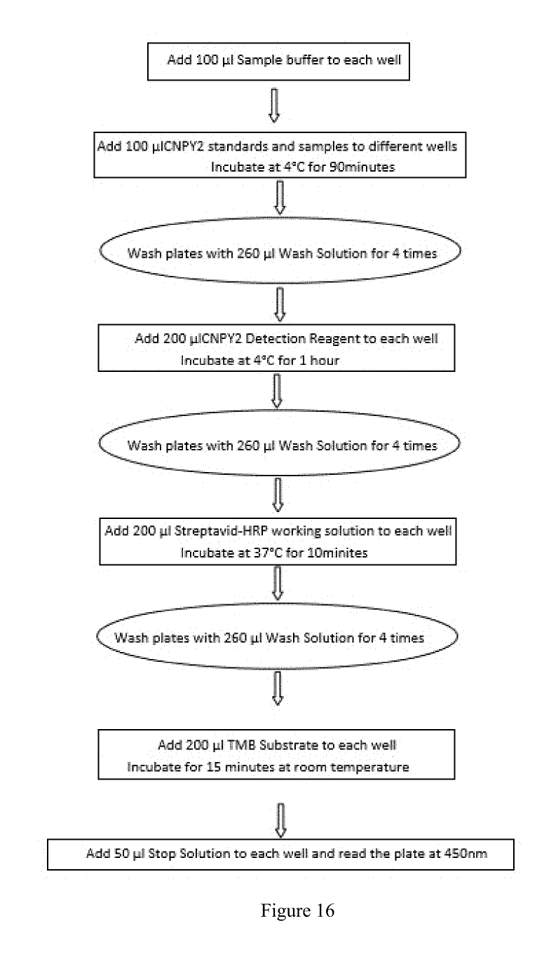

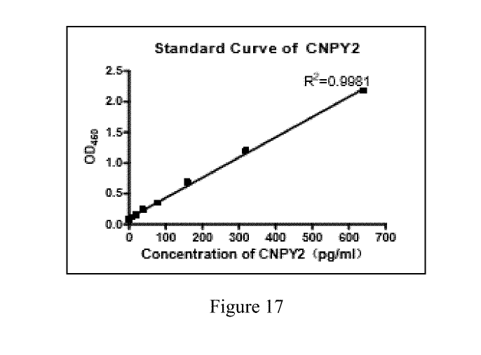

See FIG. 16 for detailed detection procedure

7. Reference Curve

The curve in FIG. 17 is an example, and a standard curve should be prepared in each of detection.

TABLE 7

| CNPY2 Standard | OD450 | | |
|---|---|---|---|
| (pg/mL) | Repetition 1 | Repetition 2 | Mean Value |
| 640 | 2.283 | 2.077 | 2.180 |
| 320 | 1.241 | 1.156 | 1.199 |
| 160 | 0.68 | 0.69 | 0.685 |
| 80 | 0.364 | 0.338 | 0.351 |
| 40 | 0.246 | 0.224 | 0.235 |
| 20 | 0.146 | 0.155 | 0.151 |
| 10 | 0.116 | 0.115 | 0.116 |
| 0 | 0.078 | 0.077 | 0.078 |

8. Sensitivity

The kit has a sensitivity of 2.369 pg/ml (mean value of four tests). The sensitivity is the concentration corresponding to the sum of mean OD450 of 20 zero standard well and triple standard deviation.

9. Precision

The intra-batch average coefficient of variation of the kit is lower than 5%, and the inter-batch average coefficient of variation is lower than 10%.

Intra-batch precision: Three different levels of CNPY2 were tested 10 times on one plate to assess intra-assay precision.

Inter-batch precision: Three different levels of CNPY2 were tested in 4 separate assays to assess inter-assay precision.

TABLE 8

| Intra-batch difference | | | | Inter-batch difference | | | |
|---|---|---|---|---|---|---|---|
| | Average | | Coefficient | | Average | | Coefficient |
| Set | value | | of | Set | value | | of |
| value | detected | | variation | value | detected | | variation |
| (pg/ml) | (pg/ml) | SD | (%) | (pg/ml) | (pg/ml) | SD | (%) |
| 320 | 343.62 | 15.317 | 4.46% | 320 | 288.15 | 28.77 | 9.98% |
| 160 | 173.93 | 7.720 | 4.44% | 160 | 158.73 | 1.92 | 1.21% |
| 40 | 35.39 | 1.961 | 5.54% | 40 | 36.85 | 2.34 | 6.34% |

10. Recovery

Three different concentrations of CNPY2 were added into human serum and plasma, and the recovery is as follows:

TABLE 9

| Dilution | | Human serum | Human plasma |
|---|---|---|---|
| ratio | N/A | (n = 3) | (n = 3) |
| No dilution | Average recovery | 43.6% | 66.2% |
| | (%) | | |
| | Range (%) | 33.9%-54.3% | 56.1%-78.7% |
| 1:2 | Average recovery | 60.1% | 81.3% |
| | (%) | | |
| | Range (%) | 54.7%-68.2% | 72.7%-89.8% |
| 1:5 | Average recovery | 86.2% | 112.5% |
| | (%) | | |
| | Range (%) | 82.3%-92.7% | 90.2%-122.0% |
| 1:10 | Average recovery | 89.1% | 107.7% |
| | (%) | | |
| | Range (%) | 82.3%-95.5% | 96.6%-117.0% |

According to the result of recovery, the Serum and Plasma samples are suggested diluted by more than 1:5.

TABLE 10
PCR raw data of mRNA level in intestinal cancer patients

| | | | | | | | | | | | CNPY2 | | GAPDH | | |
|---|---|---|---|---|---|---|---|---|---|---|---|---|---|---|---|
| | | | | | | | | | | | fold | | fold | | |
| | | | Path- | | | | | | | | change | | change | | |
| Data | | TMN | ological | | | Cancer- | Normal- | Cancer- | Normal- | Δ CT | (cancer/ | Δ CT | (cancer/ | | |
| No. | Diagnosis | stage | stage | Age | Gender | CNPY2 | CNPY2 | GAPDH | GAPDH | CNPY2 | normal) | GAPDH | normal) | Δ-Δ CT | FC |
| 147 | sigmoid | T2N0M0 | 1 | 73 | male | 25.35666667 | 30.51333333 | 16.71 | 20.25 | 5.156666667 | 36 | 3.54 | 12 | 1.616666667 | 3.066656689 |
| | colon | | | | | | | | | | | | | | |
| | cancer | | | | | | | | | | | | | | |
| 222 | ascending | T2N0M0 | 1 | 49 | female | 23.49333333 | 30.19666667 | 15.21 | 21.53333333 | 6.703333333 | 104 | 6.323333333 | 80 | 0.38 | 1.301341855 |
| | colon | | | | | | | | | | | | | | |
| | cancer | | | | | | | | | | | | | | |
| 273 | sigmoid | T2N0M0 | 1 | 59 | male | 24.18333333 | 30.82666667 | 16.29333333 | 21.24666667 | 6.643333333 | 100 | 4.953333333 | 31 | 1.69 | 3.226567037 |
| | colon | | | | | | | | | | | | | | |
| | cancer | | | | | | | | | | | | | | |
| 310 | sigmoid | T2N0M0 | 1 | 65 | female | 26.54666667 | 28.56333333 | 18.48666667 | 18.95666667 | 2.016666667 | 4 | 0.47 | 1 | 1.546666667 | 2.921413689 |
| | colon | | | | | | | | | | | | | | |
| | cancer | | | | | | | | | | | | | | |
| 376 | sigmoid | T2N0M0 | 1 | 60 | male | 23.54333333 | 28.09 | 15.12333333 | 19.34666667 | 4.546666667 | 23 | 4.223333333 | 19 | 0.323333333 | 1.251218139 |
| | colon | | | | | | | | | | | | | | |
| | cancer | | | | | | | | | | | | | | |
| 128 | rectal | T2N0M0 | 1 | 57 | female | 32.06333333 | 30.38 | 19.52333333 | 20.38 | −1.683333333 | 0 | 0.856666667 | 2 | −2.54 | 0.171942727 |
| | cancer | | | | | | | | | | | | | | |
| 195 | rectal | T2N0M0 | 1 | 50 | female | 24.02 | 31.4 | 15.18 | 20.2 | 7.38 | 167 | 5.02 | 32 | 2.36 | 5.13370359 |
| | cancer | | | | | | | | | | | | | | |
| 204 | rectal | T2N0M0 | 1 | 58 | female | 25.99333333 | 30.95333333 | 16.86333333 | 19.95333333 | 4.96 | 31 | 3.09 | 9 | 1.87 | 3.655325801 |
| | cancer | | | | | | | | | | | | | | |
| 230 | rectal | T2N0M0 | 1 | 55 | male | 24.04333333 | 27.83333333 | 14.26333333 | 18.91666667 | 3.79 | 14 | 4.653333333 | 25 | −0.863333333 | 0.549681057 |
| | cancer | | | | | | | | | | | | | | |
| 315 | rectal | T2N0M0 | 1 | 75 | male | 25.73666667 | 32.815 | 16.81666667 | 19.88666667 | 7.078333333 | 135 | 3.07 | 8 | 4.008333333 | 16.09268706 |
| | cancer | | | | | | | | | | | | | | |
| 118 | ascending | T3N0M0 | 2 | 57 | male | 30.68666667 | 24.83666667 | 21.73 | 15.71333333 | −5.85 | 0 | −6.016666667 | 0 | 0.166666667 | 1.122462048 |
| | colon | | | | | | | | | | | | | | |
| | cancer | | | | | | | | | | | | | | |
| 130 | ascending | T3N0M0 | 2 | 70 | female | 26.40333333 | 28.95 | 16.94666667 | 19.16333333 | 2.546666667 | 6 | 2.216666667 | 5 | 0.33 | 1.257013375 |
| | colon | | | | | | | | | | | | | | |
| | cancer | | | | | | | | | | | | | | |
| 196 | transverse | T3N0M0 | 2 | 30 | female | 24.09666667 | 30.69666667 | 16.14333333 | 21.63666667 | 6.6 | 97 | 5.493333333 | 45 | 1.106666667 | 2.153475136 |
| | colon | | | | | | | | | | | | | | |
| | cancer | | | | | | | | | | | | | | |
| 219 | sigmoid | T4N0M0 | 2 | 62 | male | 26.95 | 30.14 | 17.44333333 | 21.07 | 3.19 | 9 | 3.626666667 | 12 | −0.436666667 | 0.73883972 |
| | colon | | | | | | | | | | | | | | |
| | cancer | | | | | | | | | | | | | | |
| 174 | rectal | T3N0M0 | 2 | 51 | male | 29.10333333 | 29.8 | 20.01333333 | 19.64333333 | 0.696666667 | 2 | −0.37 | 1 | 1.066666667 | 2.094588246 |
| | cancer | | | | | | | | | | | | | | |
| 177 | rectal | T4N0M0 | 2 | 39 | male | 22.98666667 | 27.56 | 15.89 | 17.90333333 | 4.573333333 | 24 | 2.013333333 | 4 | 2.56 | 5.897076869 |
| | cancer | | | | | | | | | | | | | | |
| 194 | rectal | T4N0M0 | 2 | 59 | male | 24.96666667 | 29.58 | 17.11333333 | 20.28 | 4.613333333 | 24 | 3.166666667 | 9 | 1.446666667 | 2.725775354 |
| | cancer | | | | | | | | | | | | | | |
| 282 | rectal | T4N0M0 | 2 | 58 | female | 26.53333333 | 30.81666667 | 18.28 | 20.55666667 | 4.283333333 | 19 | 2.276666667 | 5 | 2.006666667 | 4.018526698 |
| | cancer | | | | | | | | | | | | | | |
| 146 | transverse | T3N1M0 | 3 | 58 | male | 24.01666667 | 30.17 | 14.71666667 | 20.82333333 | 6.153333333 | 71 | 6.106666667 | 69 | 0.046666667 | 1.032875715 |
| | colon | | | | | | | | | | | | | | |
| | cancer | | | | | | | | | | | | | | |
| 154 | sigmoid | T3N1M0 | 3 | 65 | male | 31.33 | 29.9 | 21.02 | 20.80333333 | −1.43 | 0 | −0.216666667 | 1 | −1.213333333 | 0.431271016 |
| | colon | | | | | | | | | | | | | | |
| | cancer | | | | | | | | | | | | | | |
| 161 | sigmoid | T3N1M0 | 3 | 68 | male | 24.57333333 | 29.58 | 17.02666667 | 20.04 | 5.006666667 | 32 | 3.013333333 | 8 | 1.993333333 | 3.981558716 |
| | colon | | | | | | | | | | | | | | |
| | cancer | | | | | | | | | | | | | | |
| 191 | sigmoid | T3N1M0 | 3 | 46 | male | 25.12 | 27.62 | 16.86333333 | 18.7 | 2.5 | 6 | 1.836666667 | 4 | 0.663333333 | 1.583737611 |
| | colon | | | | | | | | | | | | | | |
| | cancer | | | | | | | | | | | | | | |
| 201 | descending | T3N1M0 | 3 | 36 | male | 27.00333333 | 30.25 | 17.05 | 21.42666667 | 3.246666667 | 9 | 4.376666667 | 21 | −1.13 | 0.456915725 |
| | colon | | | | | | | | | | | | | | |
| | cancer | | | | | | | | | | | | | | |
| 264 | sigmoid | T2N1M0 | 3 | 57 | female | 27.02666667 | 28.03666667 | 18.26666667 | 19.04 | 1.01 | 2 | 0.773333333 | 2 | 0.236666667 | 1.178267139 |
| | colon | | | | | | | | | | | | | | |
| | cancer | | | | | | | | | | | | | | |
| 321 | transverse | T3N1M0 | 3 | 68 | female | 24.70333333 | 31.38333333 | 15.63333333 | 22.18666667 | 6.68 | 103 | 6.553333333 | 94 | 0.126666667 | 1.091768265 |
| | colon | | | | | | | | | | | | | | |
| | cancer | | | | | | | | | | | | | | |
| 338 | transverse | T3N2M0 | 3 | 65 | female | 24.22 | 28.63 | 15.60333333 | 19.18333333 | 4.41 | 21 | 3.58 | 12 | 0.83 | 1.777685362 |
| | colon | | | | | | | | | | | | | | |
| | cancer | | | | | | | | | | | | | | |
| 378 | ascending | T4N1M0 | 3 | 43 | female | 28.42333333 | 40 | 18.88 | 21.70333333 | 11.57666667 | 3054 | 2.823333333 | 7 | 8.753333333 | 431.5348709 |
| | colon | | | | | | | | | | | | | | |
| | cancer | | | | | | | | | | | | | | |
| 120 | rectal | T2N1M0 | 3 | 41 | female | 24.33666667 | 34.63666667 | 15.04666667 | 21.93333333 | 10.3 | 1261 | 6.886666667 | 118 | 3.413333333 | 10.65407424 |
| | cancer | | | | | | | | | | | | | | |
| 125 | rectal | T3N1M0 | 3 | 66 | female | 25.03333333 | 30.20666667 | 15.48333333 | 19.41333333 | 5.173333333 | 36 | 3.93 | 15 | 1.243333333 | 2.367448977 |
| | cancer | | | | | | | | | | | | | | |
| 132 | rectal | T4N1M0 | 3 | 33 | female | 26.16333333 | 30.09666667 | 18.07 | 20.92333333 | 3.933333333 | 15 | 2.853333333 | 7 | 1.08 | 2.114036081 |
| | cancer | | | | | | | | | | | | | | |
| 176 | rectal | T4N1M0 | 3 | 57 | male | 27.56333333 | 28.87333333 | 18.94666667 | 19.11333333 | 1.31 | 2 | 0.166666667 | 1 | 1.143333333 | 2.208908001 |
| | cancer | | | | | | | | | | | | | | |
| 197 | rectal | T4N1M0 | 3 | 59 | male | 26.74333333 | 31.92333333 | 18.35 | 21.07333333 | 5.18 | 36 | 2.723333333 | 7 | 2.456666667 | 5.489469242 |
| | cancer | | | | | | | | | | | | | | |
| 207 | rectal | T4N1M0 | 3 | 70 | male | 23.69666667 | 30.47 | 14.94333333 | 21.06666667 | 6.773333333 | 109 | 6.123333333 | 70 | 0.65 | 1.569168196 |
| | cancer | | | | | | | | | | | | | | |
| 216 | rectal | T2N1M0 | 3 | 73 | female | 23.80333333 | 29.67 | 15.63 | 20.12333333 | 5.866666667 | 58 | 4.493333333 | 23 | 1.373333333 | 2.590684504 |
| | cancer | | | | | | | | | | | | | | |
| 240 | rectal | T4N1M0 | 3 | 60 | male | 27.00333333 | 28.83666667 | 18.92 | 19.27333333 | 1.833333333 | 4 | 0.353333333 | 1 | 1.48 | 2.789487333 |
| | cancer | | | | | | | | | | | | | | |
| 296 | rectal | T3N1M0 | 3 | 36 | male | 26.09666667 | 31.12 | 17.61 | 20.64333333 | 5.023333333 | 33 | 3.033333333 | 8 | 1.99 | 3.972369982 |
| | cancer | | | | | | | | | | | | | | |
| 301 | rectal | T4N1M0 | 3 | 74 | male | 26.26333333 | 28.52666667 | 16.47 | 18.83666667 | 2.263333333 | 5 | 2.366666667 | 5 | −0.103333333 | 0.930879716 |
| | cancer | | | | | | | | | | | | | | |
| 131 | transverse | T4N0M1 | 4 | 55 | male | 25.53333333 | 30.46333333 | 16.66 | 22.68333333 | 4.93 | 30 | 6.023333333 | 65 | −1.093333333 | 0.468677248 |
| | colon | | | | | | | | | | | | | | |
| | cancer | | | | | | | | | | | | | | |
| 135 | sigmoid | T4N1M1 | 4 | 59 | female | 24.93333333 | 28.06333333 | 16.48 | 18.49 | 3.13 | 9 | 2.01 | 4 | 1.12 | 2.173469725 |
| | colon | | | | | | | | | | | | | | |
| | cancer | | | | | | | | | | | | | | |
| 139 | sigmoid | T3N0M1 | 4 | 63 | male | 23.66 | 27.44666667 | 15.2 | 19.08333333 | 3.786666667 | 14 | 3.883333333 | 15 | −0.096666667 | 0.935191248 |
| | colon | | | | | | | | | | | | | | |
| | cancer | | | | | | | | | | | | | | |
| 148 | sigmoid | T4N1M1 | 4 | 48 | male | 23.94333333 | 27.71 | 15.38 | 18.52333333 | 3.766666667 | 14 | 3.143333333 | 9 | 0.623333333 | 1.540430222 |
| | colon | | | | | | | | | | | | | | |
| | cancer | | | | | | | | | | | | | | |
| 193 | ileocecal | T4N1M1 | 4 | 64 | female | 31.07 | 27.20333333 | 19.77 | 18.45666667 | −3.866666667 | 0 | −1.313333333 | 0 | −2.553333333 | 0.17036096 |
| | cancer | | | | | | | | | | | | | | |
| 245 | ascending | T4N1M1 | 4 | 61 | female | 23.75 | 28.66 | 14.33666667 | 19.40666667 | 4.91 | 30 | 5.07 | 34 | −0.16 | 0.895025071 |
| | colon | | | | | | | | | | | | | | |
| | cancer | | | | | | | | | | | | | | |
| 255 | sigmoid | T3N0M1 | 4 | 60 | female | 23.84333333 | 29.00333333 | 15.18666667 | 19.68666667 | 5.16 | 36 | 4.5 | 23 | 0.66 | 1.580082624 |
| | colon | | | | | | | | | | | | | | |
| | cancer | | | | | | | | | | | | | | |
| 272 | sigmoid | T3N2M1 | 4 | 43 | male | 29.035 | 29.44 | 19.76333333 | 19.85333333 | 0.405 | 1 | 0.09 | 1 | 0.315 | 1.244011653 |
| | colon | | | | | | | | | | | | | | |
| | cancer | | | | | | | | | | | | | | |
| 355 | sigmoid | T3N0M1 | 4 | 63 | male | 24.67333333 | 28.655 | 15.94333333 | 19.64333333 | 3.981666667 | 16 | 3.7 | 13 | 0.281666667 | 1.215598388 |
| | colon | | | | | | | | | | | | | | |
| | cancer | | | | | | | | | | | | | | |
| 368 | sigmoid | T3N2M1 | 4 | 66 | male | 25.76333333 | 28.88333333 | 17.495 | 19.69333333 | 3.12 | 9 | 2.198333333 | 5 | 0.921666667 | 1.894302414 |
| | colon | | | | | | | | | | | | | | |
| | cancer | | | | | | | | | | | | | | |
| 110 | rectal | T4N1M1 | 4 | 49 | female | 27.23333333 | 28.88666667 | 18.19333333 | 18.04333333 | 1.653333333 | 3 | −0.15 | 1 | 1.803333333 | 3.490257151 |
| | cancer | | | | | | | | | | | | | | |
| 137 | rectal | T4N1M1 | 4 | 48 | male | 27.53333333 | 28.19666667 | 17.81 | 18.11 | 0.663333333 | 2 | 0.3 | 1 | 0.363333333 | 1.286394669 |
| | cancer | | | | | | | | | | | | | | |
| 180 | rectal | T4N0M1 | 4 | 65 | male | 24.65333333 | 33 | 15.64666667 | 20.96333333 | 8.346666667 | 326 | 5.316666667 | 40 | 3.03 | 8.168097006 |
| | cancer | | | | | | | | | | | | | | |
| 279 | rectal | T4N0M1 | 4 | 49 | male | 25.95 | 28.48333333 | 17.46666667 | 19.30666667 | 2.533333333 | 6 | 1.84 | 4 | 0.693333333 | 1.617015304 |
| | cancer | | | | | | | | | | | | | | |
| 304 | rectal | T4N0M1 | 4 | 51 | female | 30.855 | 28.34666667 | 19.96333333 | 18.88333333 | −2.508333333 | 0 | −1.08 | 0 | −1.428333333 | 0.371559888 |
| | cancer | | | | | | | | | | | | | | |
| 397 | rectal | T4N1M1 | 4 | 59 | male | 24.97333333 | 29.61333333 | 16.06 | 20.46 | 4.64 | 25 | 4.4 | 21 | 0.24 | 1.180992661 |
| | cancer | | | | | | | | | | | | | | |
| 427 | rectal | T4N2M1 | 4 | 51 | male | 28.29 | 32.56333333 | 19.04333333 | 21.51666667 | 4.273333333 | 19 | 2.473333333 | 6 | 1.8 | 3.482202253 |
| | cancer | | | | | | | | | | | | | | |
| 528 | rectal | T3N2M1 | 4 | 73 | male | 27.91666667 | 26.48666667 | 18.63333333 | 17.55666667 | −1.43 | 0 | −1.076666667 | 0 | −0.353333333 | 0.782773416 |
| | cancer | | | | | | | | | | | | | | |
| 608 | rectal | T3N1M1 | 4 | 62 | male | 25.37 | 28.21666667 | 15.94666667 | 19.18666667 | 2.846666667 | 7 | 3.24 | 9 | −0.393333333 | 0.761368436 |
| | cancer | | | | | | | | | | | | | | |
| 692 | rectal | T4N1M1 | 4 | 43 | male | 23.91666667 | 29.69 | 15.63666667 | 20.64333333 | 5.773333333 | 55 | 5.006666667 | 32 | 0.766666667 | 1.701334322 |
| | cancer | | | | | | | | | | | | | | |
| | and its | | | | | | | | | | | | | | |
| | hepatic | | | | | | | | | | | | | | |
| | metastasis | | | | | | | | | | | | | | |
| | | | | | | 1482.723333 | 1692.893333 | | | | | | | | |
| | | | | | Sum of | 1482.723333 | 1692.893333 | 973.175 | 1133.133333 | | | | | | |
| | | | | | ct | | | | | | | | | | |
| | | | | | Average | 26.01269006 | 29.69988304 | 17.07324561 | 19.87953216 | | | | | | |
| | | | | | of ct | | | | | | | | | | |
| | | | | | Average | 12.88118114 | | 6.994818144 | | | | | | | |
| | | | | | of FC | | | | | | | | | | |

TABLE 11

| Immunohistochemistry results of tissue slice of | | | |
|---|---|---|---|
| intestinal cancer patient | | | |
| | | Normal | Intestinal |
| No. | Stage | tissues | cancer tissues |
| 1-1 | 1 | | 8.00 |
| 1-2 | 1 | 1.00 | 10.67 |
| 1-3 | 1 | 1.50 | 8.00 |
| 1-4 | 1 | 3.00 | |
| 1-5 | 1 | | 8.00 |
| 1-6 | 1 | | 7.33 |
| 1-7 | 1 | 2.00 | 10.00 |
| 1-8 | 1 | 2.00 | 8.67 |
| 1-9 | 1 | | 2.00 |
| 1-10 | 1 | | 6.33 |
| 1-11 | 1 | | |
| 1-12 | 1 | 1.67 | 6.00 |
| 1-13 | 1 | 2.00 | 9.33 |
| 1-14 | 1 | 1.50 | 9.00 |
| 1-15 | 1 | 0.00 | 3.33 |
| 1-16 | 1 | | 3.50 |
| 1-17 | 1 | | 5.50 |
| 1-18 | 1 | 0.00 | 8.00 |
| 1-19 | 1 | | 8.00 |
| 1-20 | 1 | | 12.00 |
| 1-21 | 1 | 2.33 | 7.33 |
| 1-22 | 1 | 3.00 | 8.00 |
| 1-23 | 1 | 0.50 | 12.00 |
| 1-24 | 1 | 0.00 | 8.00 |
| 1-25 | 1 | 0.25 | 8.00 |
| 1-26 | 1 | 0.00 | 9.33 |
| 1-27 | 1 | 3.00 | |
| 1-28 | 1 | 1.00 | 9.33 |
| 1-29 | 1 | 0.50 | 12.00 |
| 1-30 | 1 | 0.00 | 8.00 |
| 1-31 | 1 | 0.00 | 8.00 |
| 1-32 | 1 | 1.33 | 10.67 |
| 1-33 | 1 | 0.00 | 6.67 |
| 1-34 | 1 | 3.00 | 12.00 |
| 1-35 | 1 | 5.50 | 12.00 |
| 1-36 | 1 | | |
| 1-37 | 1 | 1.50 | 10.67 |
| 1-38 | 1 | 3.00 | 8.00 |
| 1-39 | 1 | 1.67 | 12.00 |
| 1-40 | 1 | 4.00 | 9.33 |
| 1-41 | 1 | 0.75 | 10.67 |
| 1-42 | 1 | 2.50 | 8.00 |
| 1-43 | 1 | 2.00 | 8.00 |
| 1-44 | 1 | | 8.00 |
| 1-45 | 1 | 2.00 | 8.00 |
| 1-46 | 1 | 3.00 | 6.00 |
| 1-47 | 1 | | 8.00 |
| 1-48 | 1 | 0.00 | 5.33 |
| 1-49 | 1 | 1.00 | 3.00 |
| 1-50 | 1 | 0.00 | 6.00 |
| 1-51 | 1 | | 3.00 |
| 1-52 | 1 | 1.00 | |
| 1-53 | 1 | 2.50 | 12.00 |
| 1-54 | 1 | 1.33 | 5.33 |
| 1-55 | 1 | 0.50 | 8.00 |
| 1-56 | 1 | 0.00 | 5.33 |
| 1-57 | 1 | 0.00 | 10.67 |
| 1-58 | 1 | 0.00 | 10.00 |
| 1-59 | 1 | 1.50 | 9.33 |
| 1-60 | 1 | 2.00 | 5.33 |
| 1-61 | 1 | 0.00 | 10.67 |
| 1-62 | 1 | 0.00 | 12.00 |
| 1-63 | 1 | 1.00 | 6.67 |
| 1-64 | 1 | 0.00 | 8.00 |
| 1-65 | 1 | | 3.00 |
| 1-66 | 1 | 2.00 | 10.00 |
| 1-67 | 1 | | 8.00 |
| | Mean | 1.34 | 8.12 |
| | SD | 1.26 | 2.56 |
| | P Value | 0.0000000000 | |
| No. | Stage | normal tissues | tumor tissues |
| 2-1 | 2 | 4.00 | |
| 2-2 | 2 | 0.50 | 4.00 |
| 2-3 | 2 | 0.00 | 3.67 |
| 2-4 | 2 | | 1.33 |
| 2-5 | 2 | | 5.50 |
| 2-6 | 2 | 0.00 | 8.00 |
| 2-7 | 2 | 0.00 | 4.00 |
| 2-8 | 2 | 3.00 | 2.00 |
| 2-9 | 2 | 1.50 | 5.40 |
| 2-10 | 2 | 3.50 | 3.00 |
| 2-11 | 2 | 5.33 | 3.50 |
| 2-12 | 2 | 3.00 | 9.33 |
| 2-13 | 2 | | 12.00 |
| 2-14 | 2 | 0.00 | 4.67 |
| 2-15 | 2 | 1.50 | 8.00 |
| 2-16 | 2 | | 9.33 |
| 2-17 | 2 | 4.00 | |
| 2-18 | 2 | | 8.00 |
| 2-19 | 2 | 0.00 | 10.00 |
| 2-20 | 2 | | |
| 2-21 | 2 | | 12.00 |
| 2-22 | 2 | 0.00 | 8.67 |
| 2-23 | 2 | 1.00 | 9.00 |
| 2-24 | 2 | | 9.33 |
| 2-25 | 2 | 3.00 | 9.00 |
| 2-26 | 2 | 3.00 | 9.33 |
| 2-27 | 2 | | 12.00 |
| 2-28 | 2 | 0.00 | 10.67 |
| 2-29 | 2 | 0.00 | 4.00 |
| 2-30 | 2 | 1.00 | 2.50 |
| 2-31 | 2 | 0.00 | 6.67 |
| 2-32 | 2 | 0.00 | 9.00 |
| 2-33 | 2 | 2.00 | 8.00 |
| 2-34 | 2 | | 8.00 |
| 2-35 | 2 | 3.00 | 5.33 |
| 2-36 | 2 | 0.00 | 6.33 |
| 2-37 | 2 | 0.00 | 12.00 |
| 2-38 | 2 | 0.00 | 6.33 |
| 2-39 | 2 | | 10.00 |
| 2-40 | 2 | | 7.33 |
| 2-41 | 2 | | 8.00 |
| 2-42 | 2 | | 6.00 |
| 2-43 | 2 | | 8.67 |
| 2-44 | 2 | 4.00 | 12.00 |
| 2-45 | 2 | 0.00 | 6.00 |
| 2-46 | 2 | | 12.00 |
| 2-47 | 2 | 1.80 | |
| 2-48 | 2 | 3.00 | 11.00 |
| 2-49 | 2 | 1.50 | 9.50 |
| 2-50 | 2 | 3.00 | 9.33 |
| 2-51 | 2 | | 12.00 |
| 2-52 | 2 | | 10.00 |
| 2-53 | 2 | 0.75 | 10.67 |
| 2-54 | 2 | | 10.67 |
| 2-55 | 2 | 2.00 | 8.00 |
| 2-56 | 2 | | 10.67 |
| 2-57 | 2 | 0.00 | 8.75 |
| 2-58 | 2 | 2.33 | 5.50 |
| 2-59 | 2 | 0.60 | 4.00 |
| 2-60 | 2 | 0.00 | 4.67 |
| 2-61 | 2 | 1.50 | 8.00 |
| 2-62 | 2 | 2.67 | 12.00 |
| 2-63 | 2 | 3.00 | 12.00 |
| 2-64 | 2 | | 8.00 |
| 2-65 | 2 | 0.00 | 10.67 |
| 2-66 | 2 | 0.00 | 1.00 |
| 2-67 | 2 | | 6.67 |
| 2-68 | 2 | 1.33 | 12.00 |
| 2-69 | 2 | 4.00 | 3.00 |
| 2-70 | 2 | 0.00 | 3.33 |
| 2-71 | 2 | 0.00 | 5.00 |
| 2-72 | 2 | 1.75 | 11.00 |
| 2-73 | 2 | 0.00 | 12.00 |
| 2-74 | 2 | 0.00 | 7.33 |
| 2-75 | 2 | 0.00 | 5.00 |
| 2-76 | 2 | | |
| 2-77 | 2 | 2.00 | 8.00 |
| 2-78 | 2 | 0.00 | 8.00 |
| 2-79 | 2 | 0.00 | 5.50 |
| 2-80 | 2 | | 3.33 |
| 2-81 | 2 | | 5.00 |
| 2-82 | 2 | 0.00 | 10.67 |
| 2-83 | 2 | 1.00 | 6.67 |
| 2-84 | 2 | 0.00 | 12.00 |
| 2-85 | 2 | 0.00 | 9.00 |
| 2-86 | 2 | 0.00 | 7.33 |
| 2-87 | 2 | 2.50 | 10.67 |
| 2-88 | 2 | | 0.00 |
| 2-89 | 2 | 0.00 | 11.20 |
| 2-90 | 2 | 0.00 | 9.33 |
| 2-91 | 2 | | |
| 2-92 | 2 | 1.50 | 8.00 |
| 2-93 | 2 | 0.00 | 7.33 |
| 2-94 | 2 | 0.00 | 0.00 |
| 2-95 | 2 | 3.00 | 11.00 |
| 2-96 | 2 | 0.00 | 3.33 |
| 2-97 | 2 | 2.00 | 6.00 |
| 2-98 | 2 | 0.00 | 6.67 |
| 2-99 | 2 | | 12.00 |
| 2-100 | 2 | 0.00 | 8.00 |
| 2-101 | 2 | 0.00 | 5.67 |
| 2-102 | 2 | | 2.50 |
| 2-103 | 2 | 0.00 | 9.33 |
| 2-104 | 2 | 0.00 | 6.00 |
| 2-105 | 2 | | 6.67 |
| 2-106 | 2 | 0.00 | 8.00 |
| 2-107 | 2 | 2.00 | 8.00 |
| 2-108 | 2 | 0.00 | |
| 2-109 | 2 | 3.00 | |
| 2-110 | 2 | 0.50 | 4.50 |
| 2-111 | 2 | | 8.00 |
| 2-112 | 2 | 4.67 | |
| 2-113 | 2 | 1.00 | 5.50 |
| | Mean | 1.17 | 7.55 |
| | SD | 1.44 | 3.08 |
| | P Value | 0.0000000000 | |
| 4-1 | 4 | 2.50 | |
| 4-2 | 4 | | 2.67 |
| 4-3 | 4 | | 4.00 |
| 4-4 | 4 | 0.00 | 12.00 |
| 4-5 | 4 | | 9.33 |
| 4-6 | 4 | | 8.00 |
| 4-7 | 4 | 4.25 | 12.00 |
| 4-8 | 4 | | |
| 4-9 | 4 | 3.50 | |
| 4-10 | 4 | | |
| 4-11 | 4 | 1.50 | 9.33 |
| 4-12 | 4 | 0.00 | 9.33 |
| 4-13 | 4 | 3.67 | |
| 4-14 | 4 | 3.00 | |
| 4-15 | 4 | | |
| 4-16 | 4 | | 0.00 |
| 4-17 | 4 | 0.75 | 12.00 |
| 4-18 | 4 | 4.00 | |
| 4-19 | 4 | 3.00 | 8.00 |
| 4-20 | 4 | | |
| 4-21 | 4 | | 9.33 |
| 4-22 | 4 | | 9.33 |
| 4-23 | 4 | | 3.00 |
| 4-24 | 4 | 0.50 | 6.00 |
| 4-25 | 4 | 2.75 | 8.00 |
| 4-26 | 4 | 0.00 | 6.67 |
| 4-27 | 4 | | 6.33 |
| 4-28 | 4 | 2.50 | 8.00 |
| 4-29 | 4 | 0.50 | 8.00 |
| 4-30 | 4 | | 6.67 |
| 4-31 | 4 | 3.50 | 4.00 |
| 4-32 | 4 | 2.50 | 6.67 |
| 4-33 | 4 | | |
| 4-34 | 4 | 1.00 | 4.20 |
| 4-35 | 4 | 0.00 | 6.67 |
| 4-36 | 4 | 2.00 | 2.50 |
| 4-37 | 4 | 3.50 | 3.50 |
| 4-38 | 4 | 0.00 | 2.50 |
| 4-39 | 4 | 0.00 | 4.00 |
| 4-40 | 4 | | 5.50 |
| 4-41 | 4 | | 3.50 |
| 4-42 | 4 | | 8.00 |
| 4-43 | 4 | 0.00 | 3.50 |
| 4-44 | 4 | 0.00 | 3.50 |
| 4-45 | 4 | | 2.33 |
| 4-46 | 4 | | |
| 4-47 | 4 | 0.33 | 10.00 |
| 4-48 | 4 | 0.00 | 12.00 |
| 4-49 | 4 | 0.00 | 10.00 |
| 4-50 | 4 | 0.00 | 12.00 |
| 4-51 | 4 | 3.00 | |
| 4-52 | 4 | | 8.00 |
| 4-53 | 4 | 0.67 | 1.00 |
| 4-54 | 4 | 1.00 | 12.00 |
| 4-55 | 4 | 1.50 | 8.00 |
| 4-56 | 4 | 0.00 | 8.00 |
| 4-57 | 4 | 0.33 | |
| 4-58 | 4 | 4.00 | 8.00 |
| 4-59 | 4 | 3.00 | 9.33 |
| 4-60 | 4 | 4.00 | 12.00 |
| 4-61 | 4 | 2.00 | 8.00 |
| 4-62 | 4 | 0.33 | 9.00 |
| 4-63 | 4 | 0.00 | |
| 4-64 | 4 | | 8.00 |
| 4-65 | 4 | | 10.00 |
| 4-66 | 4 | | 12.00 |
| 4-67 | 4 | 1.00 | 12.00 |
| 4-68 | 4 | 0.00 | 0.00 |
| 4-69 | 4 | 0.00 | 3.67 |
| 4-70 | 4 | | |
| 4-71 | 4 | 1.00 | 6.33 |
| 4-72 | 4 | 1.00 | 8.00 |
| 4-73 | 4 | 1.50 | 6.00 |
| 4-74 | 4 | 4.00 | 9.33 |
| 4-75 | 4 | 1.00 | 9.33 |
| 4-76 | 4 | 3.00 | 12.00 |
| 4-77 | 4 | 0.67 | 10.00 |
| 4-78 | 4 | 0.00 | 12.00 |
| 4-79 | 4 | 2.00 | 10.00 |
| 4-80 | 4 | 0.00 | 5.33 |
| 4-81 | 4 | 0.00 | 8.00 |
| 4-82 | 4 | 0.00 | 5.00 |
| 4-83 | 4 | 0.00 | 6.00 |
| 4-84 | 4 | 0.00 | 6.67 |
| 4-85 | 4 | | |
| 4-86 | 4 | 0.00 | 6.67 |
| 4-87 | 4 | 0.00 | 8.00 |
| 4-88 | 4 | 0.00 | 6.33 |
| 4-89 | 4 | 0.00 | 9.33 |
| 4-90 | 4 | 0.00 | 10.00 |
| 4-91 | 4 | 0.00 | 8.00 |
| 4-92 | 4 | | 4.50 |
| 4-93 | 4 | 2.00 | 4.00 |
| 4-94 | 4 | 1.25 | 6.00 |
| 4-95 | 4 | | 6.20 |
| 4-96 | 4 | 0.00 | 5.33 |
| 4-97 | 4 | 0.00 | 4.00 |
| 4-98 | 4 | 4.00 | 12.00 |
| 4-99 | 4 | | 8.00 |
| 4-100 | 4 | 0.00 | 3.00 |
| 4-101 | 4 | 0.00 | 6.67 |
| 4-102 | 4 | 2.25 | 10.67 |
| 4-103 | 4 | 3.67 | 3.50 |
| 4-104 | 4 | 1.33 | |
| 4-105 | 4 | 2.50 | 3.67 |
| 4-106 | 4 | 0.00 | 8.00 |
| 4-107 | 4 | 4.00 | |
| 4-108 | 4 | 0.00 | 8.00 |
| 4-109 | 4 | 0.00 | 2.50 |
| 4-110 | 4 | | 8.00 |
| 4-111 | 4 | 0.00 | 2.00 |
| 4-112 | 4 | | 2.67 |
| 4-113 | 4 | 2.33 | 5.33 |
| 4-114 | 4 | | 6.67 |
| 4-115 | 4 | 3.50 | 8.00 |
| 4-116 | 4 | 1.00 | |
| 4-117 | 4 | | 9.33 |
| 4-118 | 4 | 0.00 | 8.00 |
| 4-119 | 4 | 0.50 | 8.67 |
| 4-120 | 4 | | 5.75 |
| 4-121 | 4 | 0.00 | 3.00 |
| 4-122 | 4 | 0.00 | |
| 4-123 | 4 | 1.00 | 12.00 |
| 4-124 | 4 | 1.00 | 3.00 |
| 4-125 | 4 | 3.00 | |
| 4-126 | 4 | 0.00 | 2.67 |
| 4-127 | 4 | | 7.33 |
| 4-128 | 4 | 0.00 | 9.33 |
| 4-129 | 4 | 0.00 | 8.00 |
| 4-130 | 4 | 0.00 | 4.00 |
| 4-131 | 4 | | 12.00 |
| 4-132 | 4 | 0.00 | 12.00 |
| 4-133 | 4 | 0.00 | 4.67 |
| 4-134 | 4 | 0.00 | 5.33 |
| 4-135 | 4 | 1.00 | 12.00 |
| 4-136 | 4 | 0.00 | 5.33 |
| 4-137 | 4 | 0.33 | 3.50 |
| 4-138 | 4 | | 8.00 |
| 4-139 | 4 | 0.00 | 10.67 |
| 4-140 | 4 | 1.67 | 8.00 |
| 4-141 | 4 | 0.00 | 3.50 |
| 4-142 | 4 | 1.00 | 0.00 |
| 4-143 | 4 | 0.00 | 6.33 |
| 4-144 | 4 | 2.67 | 5.00 |
| 4-145 | 4 | | 4.67 |
| 4-146 | 4 | 0.00 | 0.50 |
| 4-147 | 4 | 3.00 | 10.67 |
| 4-148 | 4 | 1.33 | 12.00 |
| 4-149 | 4 | 3.50 | 6.33 |
| 4-150 | 4 | 0.00 | 3.67 |
| 4-151 | 4 | 1.00 | 5.00 |
| 4-152 | 4 | 0.00 | 8.00 |
| 4-153 | 4 | 0.00 | 3.00 |
| 4-154 | 4 | 0.00 | 8.67 |
| 4-155 | 4 | 0.00 | 10.67 |
| 4-156 | 4 | | |
| 4-157 | 4 | 1.50 | 10.67 |
| 4-158 | 4 | 2.00 | 2.00 |
| 4-159 | 4 | 0.00 | 4.00 |
| 4-160 | 4 | 2.00 | 8.00 |
| 4-161 | 4 | 1.00 | 10.67 |
| 4-162 | 4 | 2.00 | 9.33 |
| 4-163 | 4 | | 4.00 |
| 4-164 | 4 | 0.60 | 3.50 |
| 4-165 | 4 | 4.00 | 9.33 |
| 4-166 | 4 | 1.00 | 5.33 |
| 4-167 | 4 | 0.00 | 3.00 |
| 4-168 | 4 | 0.67 | 2.50 |
| 4-169 | 4 | 0.00 | 6.00 |
| 4-170 | 4 | 0.00 | 12.00 |
| 4-171 | 4 | 0.00 | 4.00 |
| 4-172 | 4 | 1.00 | 8.00 |
| 4-173 | 4 | 0.00 | 6.67 |
| 4-174 | 4 | 0.00 | 10.67 |
| 4-175 | 4 | 2.50 | 8.00 |
| 4-176 | 4 | 0.00 | 6.00 |
| 4-177 | 4 | 0.00 | 10.67 |
| 4-178 | 4 | 0.00 | 4.00 |
| 4-179 | 4 | | |
| 4-180 | 4 | | 3.00 |
| 4-181 | 4 | 0.00 | 2.67 |
| 4-182 | 4 | 0.00 | 3.00 |
| 4-183 | 4 | 0.00 | 7.33 |
| 4-184 | 4 | 0.00 | 6.67 |
| | Mean | 1.04 | 6.86 |
| | SD | 1.33 | 3.15 |
| | P Value | 0.0000000000 | |

Hereto, those skilled in the art will recognize that while the exemplary examples of the present invention have been shown and described in details herein, it can be directly determined or derived from the disclosure of the present invention many other variations or modifications that conform to the principles of the invention without departing from the spirit and scope of the invention. Therefore, the scope of the invention should be understood and determined to cover all such other variations or modifications.

1. A molecular marker for detection of colorectal cancer, where the marker is CNPY2 protein isoform 2 having the sequence identified as SEQ ID NO:1.

2. The molecular marker according to claim 1, wherein, the mRNA sequence of the molecular marker is identified as SEQ ID NO: 2.

3. The molecular marker according to claim 1, wherein, the cDNA sequence of the molecular marker is identified as SEQ ID NO: 3.

4. A monoclonal antibody being able to specifically bind to CNPY2 protein isoform 2.

5. A kit comprising at least one antibody specifically binding to CNPY2 protein isoform 2.

6. The kit according to claim 5, comprising at least two antibodies specifically binding to CNPY2 protein isoform 2.

7. The kit according to claim 5, further comprising CNPY2 Protein Capture Plate, Sample Diluent, Sample Buffer, CNPY2 Standard, Wash Buffer, Enzyme-linked Marker and Substrate Solution.

8. The kit according to claim 7, wherein, the Enzyme-linked Marker is HRP-labeled Streptavidin, and the Substrate Solution is TMB Substrate Solution.

9. The kit according to claim 5, wherein, the kit is ELISA assay kit.

10. The kit according to claim 9, wherein, the ELISA assay kit is sandwich ELISA assay kit.

11. A medication being able to block the binding of CNPY2 protein isoform 2 to the receptor thereof, so as to prevent growth of colorectal cancer cells.

12. The medication according to claim 11, wherein, said medication blocks the binding of CNPY2 protein isoform 2 to the receptor thereof by specifically binding to CNPY2 protein isoform 2.

13. The medication according to claim 11, wherein, said medication is an antibody specifically binding to CNPY2 protein isoform 2.

14. A method for detection, diagnosis and prognosis of colorectal cancer, comprising:
a. determining concentration of protein isoform 2 from a sample of a suspected individual,
b. comparing the concentration determined in step a with that of CNPY2 protein isoform 2 from a sample of a healthy individual, as compared to the healthy individual, an increase in the concentration of CNPY2 protein isoform 2 indicates a probable presence or a risk of colorectal cancer.

15. The method according to claim 14, wherein, the individual is human, and the sample is blood, plasma or serum sample.

16. The method according to claim 14, wherein, an antibody specifically binding to CNPY2 protein isoform 2 is used to determine the concentration of CNPY2 protein isoform 2.

17. The method according to claim 14, wherein, the concentration of CNPY2 protein isoform 2 is determined by ELISA.

18. The method according to claim 14, wherein, the determined concentration of CNPY2 protein isoform 2 of the suspected individual is at least 2, times that of the healthy individual.

19. The method according to claim 14, wherein, the method further determines and compares the concentration of CNPY2 protein isoform 1 in the suspected individual and healthy individual, for combined detection, diagnosis and prognosis of colorectal cancer.

20. A method for detection, diagnosis, prognosis of colorectal cancer, comprising:
a. determining mRNA concentration of CNPY2 protein isoform 2 from a sample of a suspected individual,
b. comparing the concentration determined in step a with mRNA concentration of CNPY2 protein isoform 2 from a sample of a healthy individual, wherein, as compared to the healthy individual, an increase in the mRNA concentration of CNPY2 protein isoform 2 indicates a probable presence or a risk of colorectal cancer.

21. The method according to claim 20, wherein, the mRNA concentration of CNPY2 protein isoform 2 is determined by PCR.

22. The method according to claim 21, wherein, the PCR uses
(SEQ ID NO: 6)
a forward primer: 5′-AGACCATTCAGATGGGATCTTTC-3′,
(SEQ ID NO: 7)
a reverse primer: 5′-TTCATCCAAAGCCAGAGTGAG-3′.

23. The method according to claim 20, wherein, the determined mRNA concentration of CNPY2 protein isoform 2 of the suspected individual is at least 2, times that of the healthy individual.

24. The method according to claim 20, wherein, the method further determines and compares the mRNA concentration of CNPY2 protein isoform 1 in the detected individual and healthy individual, for combined detection, diagnosis and prognosis of colorectal cancer.

25. The method for prevention, alleviation or treatment of colorectal cancer, which comprises blocking the binding of CNPY2 protein isoform 2 to the receptor thereof.

26. The method according to claim 25, comprising administration of the antibody of claim 4 to those with a risk of colorectal cancer or colorectal cancer patients.

27. A method for prevention, alleviation or treatment of colorectal cancer, comprising reducing expression of CNPY2 protein isoform 2.


SEQUENCE LISTING (9 sequences)

SEQ ID NO: 1 - protein, 84 aa, artificial sequence
  signal peptide, CNPY protein isoform 2
MKGWGWLALLLGALLGTAWARRSQDLHCGACRALVDELEWEIAQVDPKKTIQMGSFRINPDGSQSVVEVTVTVPPNKVAHSGFG

SEQ ID NO: 2 - RNA, 1149 nt, artificial sequence
  mRNA
attgaaacctggactgctcgctggccggcagcgcaccgttttgaaggtcctagcccacctgggctggctcacgcgcacgactagccgctcccatacagcacgcccggactctgtcgtcgcttaaggccactcctattctacggctgacccctggtggtcacgtggatctgttcgccacgcaagtctgggtccttcggcgattgaccggggtccttgctgttcgggagcctctcctaagctgcctgttcgcgcgagagtttggaggggcgggtttggggtcggtgtctgattggggctcgcaccgcagcacgctggagtcccgcttaggtaccagttagcgtcaggggagctgggtcaggcggtcgccgggacaccccgtgtgtggcaggcggcgaagcgctctggagaatcccggacagccctgctccctgcagccaggtgtagtttcgggagccactggggccaaagtgagagtccagcggtcttccagcgcttgggccacggcggcggccctgggagcagaggtggagcgaccccattacgctaaagatgaaaggctggggttggctggccctgcttctgggggccctgctgggaaccgcctgggctcggaggagccaggatctccactgtggagcatgcagggctctggtggatgaactagaatgggaaattgcccaggtggaccccaagaagaccattcagatgggatctttccggatcaatccagatggcagccagtcagtggtggaggtaactgttactgttcccccaaacaaagtagctcactctggctttggatgaaattcgactgcttaaaaaggaccttggtttaatagaaatgaagaaaacagactcagaaaaaagatttggctctgtctcatttggaagaagctgcaggcttattccccatgcacttgcttcctggctgcaaaccttaatactttgtttctgctgtagaatttgttagcaaacagggagtcctgatcagcacccttctccacatccacatgactggtttttaatgtagcactgtggtatacatgcaaacatccgttcaaaatctgagtcggagctaaaaataaaaaatgaaaaaacagaaataagaataaaaggtcttatcctcataaaaaaaaaaaaaaaaaaaaa

SEQ ID NO: 3 - DNA, 255 bp, artificial sequence
  cDNA
atgaaaggctggggttggctggccctgcttctgggggccctgctgggaaccgcctgggctcggaggagccaggatctccactgtggagcatgcagggctctggtggatgaactagaatgggaaattgcccaggtggaccccaagaagaccattcagatgggatctttccggatcaatccagatggcagccagtcagtggtggaggtaactgttactgttcccccaaacaaagtagctcactctggctttggatga

SEQ ID NO: 4 - protein, 19 aa, artificial sequence
  used for ELISA screening
CVEVTVTVPPNKVAHSGFG

SEQ ID NO: 5 - protein, 18 aa, artificial sequence
  used for ELISA screening
CTIQMGSFRINPDGSQSV

SEQ ID NO: 6 - DNA, 23 bp, artificial sequence
  primer
agaccattcagatgggatctttc

SEQ ID NO: 7 - DNA, 21 bp, artificial sequence
  primer
ttcatccaaagccagagtgag

SEQ ID NO: 8 - protein, 71 aa, artificial sequence
  CNPY2 isoform 2
MRRSQDLHCGACRALVDELEWEIAQVDPKKTIQMGSFRINPDGSQSVVEVTVTVPPNKVAHSGFGHHHHHH

SEQ ID NO: 9 - protein, 35 aa, artificial sequence
  peptide
TIQMGSFRINPDGSQSVVEVTVTVPPNKVAHSGFG